(19) United States
(12) Patent Application Publication
(10) Pub. No.: US 2022/0405900 A1
(43) Pub. Date: Dec. 22, 2022

(54) INFORMATION PROCESSING DEVICE, GENERATION METHOD, AND GENERATION PROGRAM

(71) Applicant: SONY GROUP CORPORATION, TOKYO (JP)

(72) Inventors: DAISUKE KIKUCHI, TOKYO (JP); MINORI TAKAHASHI, TOKYO (JP)

(21) Appl. No.: 17/755,684

(22) PCT Filed: Nov. 9, 2020
(86) PCT No.: PCT/JP2020/041782

§ 371 Date: May 5, 2022

(30) Foreign Application Priority Data
Nov. 14, 2019 (JP) 2019-205951

(51) Int. Cl. G06T 5/50; G06T 7/00; G06T 5/00

(52) U.S. Cl. CPC G06T 5/50; G06T 7/0012; G06T 5/005; G06T 2207/10068; G06T 2207/20224; G06T 2207/20221; G06T 2207/30168; G06T 2207/30004

(57) ABSTRACT

An information processing device (100) is provided with a generation unit that acquires an input image serving as an intraoperative image and generates an output image based on whether the input image includes an intraoperatively generated matter or not.

### FIELD

The present disclosure relates to an information processing device, a generation method, and a generation program.

### Background

In medical practice, endoscope operations using an endoscope are widely practiced. Also, various devices related to endoscope operations have been developed. For example, Patent Literature 1 discloses a device which operates an insufflation device and removes smoke when the smoke is detected in a captured endoscope image.

Patent Literature 2 discloses a device which, when smoke is detected in a captured endoscope image, subjects the endoscope image to smoke removal by uniform signal processing, and then controls a smoke exhaust device in accordance with the detection result of smoke to remove the smoke.

### CITATION LIST

### Patent Literature

Patent Literature 1: JP H11-318909 A

Patent Literature 2: JP 2018-157917 A

### Summary

### Technical Problem

However, in Patent Literature 1, although the presence/absence of smoke is detected, the amount of the smoke is not detected, and the smoke cannot be sufficiently removed in some cases depending on the generation amount of the smoke. Also, in Patent Literature 1, the smoke is physically eliminated, and therefore it takes time until the smoke is exhausted and a visual field becomes clear.

In Patent Literature 2, smoke is removed from an endoscope image by uniform signal processing regardless of the generation amount of smoke, the effect of the signal processing is limited depending on the generation amount of the smoke, and the effect of the smoke removal depends on the performance of the smoke exhaust device as a result.

Therefore, the present disclosure proposes an information processing device, a generation method, and a generation program capable of reducing the influence of intraoperatively generated matters.

### Solution to Problem

To solve the problems described above, an information processing device according to an embodiment of the present disclosure includes a generation unit that acquires an input image serving as an intraoperative image and generates an output image based on whether the input image includes an intraoperatively generated matter or not.

### BRIEF DESCRIPTION OF DRAWINGS

FIG. 1 is a diagram illustrating an example of a state of an operation to which an operation room system using a technical idea according to the present disclosure is applied.

FIG. 2 is a diagram illustrating a system configuration example according to a first embodiment of the present disclosure.

FIG. 3 is a diagram illustrating a configuration example of an information processing device according to the first embodiment of the present disclosure.

FIG. 4 is a diagram for describing characteristics of smoke and mist.

FIG. 5 is a diagram illustrating relations between generation of smoke and mist and brightness and color saturation.

FIG. 6 is a diagram illustrating a configuration example of a smoke-removal processing unit according to the first embodiment of the present disclosure.

FIG. 7 is a diagram illustrating a configuration example of a mist-removal processing unit according to the first embodiment of the present disclosure.

FIG. 8 is a diagram illustrating a configuration example of a generation unit according to the first embodiment of the present disclosure.

FIG. 9 is a flow chart illustrating a flow of basic actions of the information processing device according to the first embodiment of the present disclosure.

FIG. 10 is a flow chart illustrating a flow of actions of a determination unit according to the first embodiment of the present disclosure.

FIG. 11 is a diagram illustrating a system configuration example according to a second embodiment of the present disclosure.

FIG. 12 is a diagram illustrating a configuration example of an information processing device according to the second embodiment of the present disclosure.

FIG. 13 is a diagram illustrating a configuration example of an information processing device according to a third embodiment of the present disclosure.

FIG. 14 is a diagram illustrating a configuration example of an information processing device according to a fourth embodiment of the present disclosure.

FIG. 15 is a diagram illustrating a system configuration example according to a fifth embodiment of the present disclosure.

FIG. 16 is a diagram illustrating a configuration example of an information processing device according to the fifth embodiment of the present disclosure.

FIG. 17 is a diagram illustrating an example of an output image generated by a smoke removal process according to the fifth embodiment of the present disclosure.

FIG. 18 is a diagram illustrating a configuration example of an information processing device according to a sixth embodiment of the present disclosure.

FIG. 19 is a diagram for describing a process of a superposition unit according to the sixth embodiment of the present disclosure.

FIG. 20 is a hardware configuration diagram illustrating an example of a computer which realizes functions of the information processing device.

### DESCRIPTION OF EMBODIMENTS

Hereinafter, embodiments of the present disclosure will be described in detail based on drawings. In following embodiments, the same parts are denoted by the same reference signs to omit redundant descriptions.

Also, the present disclosure is described in accordance with the order of items shown below.

1. Application Example

2. First Embodiment

2.1. Configuration of System according to First Embodiment

2.2. Configuration of Information Processing Device according to First Embodiment

2.3. Flow of Actions of Information Processing Device

2.4. Effects of Information Processing Device according to First Embodiment

3. Second Embodiment

3.1. Configuration of System according to Second Embodiment

3.2. Configuration of Information Processing Device according to Second Embodiment

3.3. Effects of Information Processing Device according to Second Embodiment

4. Third Embodiment

4.1. Configuration of System according to Third Embodiment

4.2. Configuration of Information Processing Device according to Third Embodiment

4.3. Effects of Information Processing Device according to Third Embodiment

5. Fourth Embodiment

5.1. Configuration of System according to Fourth Embodiment

5.2. Configuration of Information Processing Device according to Fourth Embodiment

5.3. Effects of Information Processing Device according to Fourth Embodiment

6. Fifth Embodiment

6.1. Configuration of System according to Fifth Embodiment

6.2. Configuration of Information Processing Device according to Fifth Embodiment

6.3. Effects of Information Processing Device according to Fifth Embodiment

7. Sixth Embodiment

7.1. Configuration of System according to Sixth Embodiment

7.2. Configuration of Information Processing Device according to Sixth Embodiment

7.3. Effects of Information Processing Device according to Sixth Embodiment

8. Hardware Configuration

9. Conclusion

### 1. APPLICATION EXAMPLE

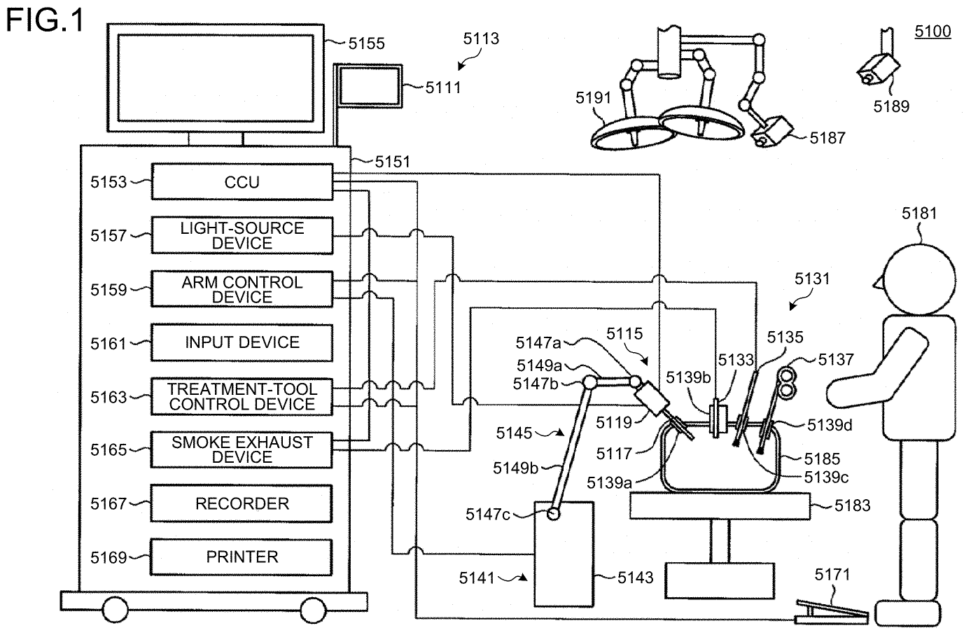

An application example of a technical idea common to embodiments of the present disclosure will be described. FIG. 1 is a diagram illustrating an example of a state of an operation to which an operation room system 5100 using the technical idea according to the present disclosure is applied. A ceiling camera 5187 and an operation site camera 5189 are provided on a ceiling of an operation room and are capable of capturing images of the state around the hands of an operator (doctor) 5181, who carries out treatment with respect to an affected part of a patient 5185 on a patient bed 5183, and the entirety of the operation room. The ceiling camera 5187 and the operation site camera 5189 can be provided with a magnification adjusting function, a focal-length adjusting function, an image-capturing-direction adjusting function, etc. A light 5191 is provided on the ceiling of the operation room and illuminates at least around the hands of the operator 5181. The light 5191 may be able to appropriately adjust, for example, an irradiation light intensity, a wavelength (color) of irradiation light, and an irradiation direction of light thereof.

An endoscope operation system 5113, the patient bed 5183, the ceiling camera 5187, the operation site camera 5189, and the light 5191 are connected so that they can work together via an audiovisual controller and an operation-room control device (not illustrated). A centralized operation panel 5111 is provided in the operation room, and a user can appropriately operate these devices, which are present in the operation room, via the centralized operation panel 5111.

Hereinafter, a configuration of the endoscope operation system 5113 will be described in detail. As illustrated, the endoscope operation system 5113 includes an endoscope 5115, other operation tools 5131, a support arm device 5141 which supports the endoscope 5115, and a cart 5151 on which various devices for an endoscope operation are mounted.

In an endoscope operation, instead of cutting the abdominal wall and carrying out a laparotomy, plural tubular aperture forming instruments called trocars 5139a to 5139d puncture the abdominal wall. Then, a lens barrel 5117 of the endoscope 5115 and other operation tools 5131 are inserted into the body cavity of the patient 5185 from the trocars 5139a to 5139d. In the illustrated example, as the other operation tools 5131, a tube 5133, an energy treatment tool 5135, and forceps 5137 are inserted in the body cavity of the patient 5185. Herein, the tube 5133 may be a structure for exhausting the smoke, which is generated in the body cavity, to outside the body cavity. Also, on the other hand, the tube 5133 may have a function to inject a gas into the body cavity and inflate the body cavity. The energy treatment tool 5135 is a treatment tool which carries out, for example, incision and scraping of tissues or sealing of blood vessels by high-frequency currents or ultrasonic oscillations. However, the illustrated operation tools 5131 are merely examples. As the operation tools 5131, for example, various operation tools generally used in endoscope operations such as pincers and retractors may be used.

An image of an operation site of the body cavity of the patient 5185 captured by the endoscope 5115 is displayed by a display device 5155. The operator 5181 carries out treatment such as removal of an affected part by using the energy treatment tool 5135 or the forceps 5137 while watching the image of the operation site displayed by the display device 5155 in real time. Although illustration is omitted, the tube 5133, the energy treatment tool 5135, and the forceps 5137 are supported, for example, by the operator 5181 or an assistant during an operation.

(Support Arm Device)

The support arm device 5141 is provided with an arm part 5145 extending from a base part 5143. In the illustrated example, the arm part 5145 includes joint parts 5147a, 5147b, and 5147c and links 5149a and 5149b and is driven by control from an arm control device 5159. The endoscope 5115 is supported by the arm part 5145, and the position and posture thereof are controlled. By virtue of this, stable position fixing of the endoscope 5115 can be realized.

(Endoscope)

The endoscope 5115 includes the lens barrel 5117 having a region, which has a predetermined length from a front end and is inserted into the body cavity of the patient 5185, and a camera head 5119, which is connected to a base end of the lens barrel 5117. In the illustrated example, the endoscope 5115 formed as a so-called hard endoscope having a hard lens barrel 5117 is illustrated. However, the endoscope 5115 may be formed as a so-called flexible endoscope having a soft lens barrel 5117.

An opening in which an objective lens fits is provided at the front end of the lens barrel 5117. A light-source device 5157 is connected to the endoscope 5115, and the light generated by the light-source device 5157 is guided to the front end of the lens barrel by a light guide, which is extending in the lens barrel 5117, and radiated toward an observation object in the body cavity of the patient 5185 via the objective lens. Note that the endoscope 5115 may be a direct-view endoscope, an oblique-view endoscope, or a side-view endoscope.

An optical system and an image capturing element are provided in the camera head 5119, and the reflected light (observation light) from the observation object is concentrated on the image capturing element by the optical system. The observation light is subjected to photoelectric conversion by the image capturing element, and an electric signal corresponding to the observation light, in other words, an image signal corresponding to the observation image is generated. The image signal is transmitted to a camera control unit (CCU: Camera Control Unit) 5153 as RAW data. The camera head 5119 is equipped with a function to adjust magnification and a focal length by appropriately driving the optical system thereof.

In order to accommodate, for example, stereoscopic view (3D display) or the like, the camera head 5119 may be provided with plural image capturing elements. In this case, in the lens barrel 5117, plural systems of relay optical systems are provided in order to guide the observation light to each of the plural image capturing elements.

(Various Devices mounted on Cart)

The CCU 5153 includes a central processing unit (CPU), a graphic processing unit (GPU), or the like and integrally controls operations of the endoscope 5115 and the display device 5155. Specifically, the CCU 5153 subjects the image signal, which has been received from the camera head 5119, to various image processing for displaying an image based on the image signal such as development processing (demosaicing processing). The CCU 5153 provides the image signal, which has undergone the image processing, to the display device 5155. Also, the above described audiovisual controller is connected to the CCU 5153. The CCU 5153 provides the image signal, which has undergone image processing, also to the audiovisual controller 5107. Also, the CCU 5153 transmits a control signal to the camera head 5119 and controls the drive thereof. The control signal can include information about image capturing conditions such as magnification and a focal length. The information about the image capturing conditions may be input via an input device 5161 or may be input via the above described centralized operation panel 5111.

The display device 5155 displays an image based on the image signal, which has been subjected to the image processing by the CCU 5153, by control from the CCU 5153. If the endoscope 5115 supports high-resolution image capturing of, for example, 4K (the number of horizontal pixels 3840×the number of vertical pixels 2160) or 8K (the number of horizontal pixels 7680×the number of vertical pixels 4320) and/or if the endoscope 5115 supports 3D display, a display device capable of carrying out high-resolution display and/or capable of carrying out 3D display is used as the display device 5155 to support it. If the endoscope is supporting high-resolution image capturing of, for example, 4K or 8K, a further sense of immersion is obtained by using a display device having a size of 55 inches or more as the display device 5155. Also, depending on use, plural display devices 5155 having different resolutions and sizes may be provided.

The light-source device 5157 includes, for example, a light source such as a light emitting diode (LED) and supplies irradiation light for image capturing of an operation site to the endoscope 5115.

The arm control device 5159 includes, for example, a processor such as a CPU, and operates in accordance with a predetermined program to control drive of the arm part 5145 of the support arm device 5141 in accordance with a predetermined control method.

The input device 5161 is an input interface for the endoscope operation system 5113. Via the input device 5161, the user can carry out input of various information or instruction input with respect to the endoscope operation system 5113. For example, via the input device 5161, the user inputs various information about the operation such as body information of the patient and information about an operation method of the operation. Also, for example, via the input device 5161, the user inputs instructions to drive the arm part 5145, instructions to change image capturing conditions (type of irradiation light, magnification, focal length, etc.) of the endoscope 5115, instructions to drive the energy treatment tool 5135, and so on.

The type of the input device 5161 is not limited, and the input device 5161 may be a publicly known input device of various types. As the input device 5161, for example, a mouse, a keyboard, a touch screen, a switch, a foot switch 5171, and/or a lever can be applied. In a case in which a touch screen is used as the input device 5161, the touch screen may be provided on a display surface of the display device 5155.

Alternatively, the input device 5161 is, for example, a device worn by the user such as an eyeglasses-type wearable device or a head mounted display (HMD), and input of various types is carried out depending on gestures and visual lines of the user detected by these devices. Also, the input device 5161 includes a camera capable of detecting the movement of the user, and input of various types is carried out depending on the gestures and visual lines of the user detected from a video captured by the camera. Furthermore, the input device 5161 includes a microphone capable of collecting the voice of the user, and input of various types is carried out by the voice via the microphone. Since the input device 5161 is configured to enable input of various information in a contactless manner in this way, particularly a user (for example, the operator 5181) belonging to a clean area can operate the equipment belonging to an unclean area in a contactless manner. Also, since the user can operate the equipment without disengaging his/her hand from an operation tool he/she is holding, user friendliness is improved.

A treatment-tool control device 5163 controls drive of the energy treatment tool 5135 for tissue cauterization, incision, blood vessel sealing, etc. A smoke exhaust device 5165 sends a gas into the body cavity via the tube 5133 to inflate the body cavity of the patient 5185 in order to ensure a visual field of the endoscope 5115 and ensure work space of the operator. Also, the smoke exhaust device 5165 has a function to exhaust the smoke which has generated in the body cavity in order to ensure the visual field of the endoscope 5115. A recorder 5167 is a device which can record various information about the operation. A printer 5169 is a device capable of printing the various information about the operation in various formats such as texts, images, or graphs.

Hereinafter, particularly characteristic structures in the endoscope operation system 5113 will be described in further detail.

(Support Arm Device)

The support arm device 5141 is provided with a base part 5143, which is a base, and the arm part 5145 extending from the base part 5143. In the illustrated example, the arm part 5145 includes the plural joint parts 5147a, 5147b, and 5147c and the plural links 5149a and 5149b coupled by the joint part 5147b. However, FIG. 1 illustrates a simplified structure of the arm part 5145 for simplicity. In practice, for example, the shape, the number, and arrangement of the joint parts 5147a to 5147c and the links 5149a and 5149b and the directions of rotation shafts of the joint parts 5147a to 5147c can be appropriately set so that the arm part 5145 has a desired degree of freedom. For example, the arm part 5145 can be preferably structured to have a degree of freedom of 6 or higher degrees of freedom. By virtue of this, the endoscope 5115 can be freely moved within a movable range of the arm part 5145. Therefore, the lens barrel 5117 of the endoscope 5115 can be inserted into the body cavity of the patient 5185 from a desired direction.

The joint parts 5147a to 5147c are provided with actuators, and the joint parts 5147a to 5147c are structured to be able to rotate about predetermined rotation axes by driving the actuators. When driving of the actuator is controlled by the arm control device 5159, rotation angles of the joint parts 5147a to 5147c are controlled, and driving of the arm part 5145 is controlled. By virtue of this, control of the position and posture of the endoscope 5115 can be realized. In this process, the arm control device 5159 can control the driving of the arm part 5145 by various publicly known control methods such as force control or position control.

For example, when the operator 5181 appropriately carries out operation input via the input device 5161 (including the foot switch 5171), the driving of the arm part 5145 may be appropriately controlled by the arm control device 5159 in accordance with the operation input to control the position and posture of the endoscope 5115. The endoscope 5115 at the front end of the arm part 5145 can be moved from an arbitrary position to another arbitrary position by this control and then can be fixedly supported at the position after the movement. Note that the arm part 5145 may be operated by a so-called master-slave method. In such a case, the arm part 5145 can be remotely operated by a user via the input device 5161, which is installed at a location distant from the operation room.

Also, in a case in which force control is applied, the arm control device 5159 may carry out so-called power-assist control which drives the actuators of the joint parts 5147a to 5147c so as to receive external force from the user and smoothly move the arm part 5145 by following the external force. As a result, when the user moves the arm part 5145 while directly touching the arm part 5145, the arm part 5145 can be moved with comparatively light force. Therefore, the endoscope 5115 can be moved more intuitively by an easier operation, and user friendliness can be improved.

Generally, in an endoscope operation, the endoscope 5115 has been supported by a doctor called a scopist. On the other hand, the position of the endoscope 5115 can be reliably fixed without manpower by using the support arm device 5141. Therefore, images of the operation site can be stably obtained, and an operation can be smoothly carried out.

Note that the arm control device 5159 is not necessarily required to be provided in the cart 5151. Also, the arm control device 5159 is not necessarily required to be one device. For example, arm control devices 5159 may be provided respectively in the joint parts 5147a to 5147c of the arm part 5145 of the support arm device 5141, and the drive control of the arm part 5145 may be realized by cooperation of the plural arm control devices 5159.

(Light-Source Device)

The light-source device 5157 supplies irradiation light to the endoscope 5115 when images of the operation site are to be captured. The light-source device 5157 includes, for example, a LED, a laser light source, or a white light source including a combination thereof. Herein, in a case in which the white light source is formed by a combination of RGB laser light sources, the white balance of captured images can be adjusted in the light-source device 5157 since the output intensity and the output timing of each color (each wavelength) can be controlled with high precision. Also, in such a case, images respectively corresponding to RGB can be also captured by time division by irradiating the observation object with the laser light from each of the RGB laser light sources by time division and controlling the driving of the image capturing element of the camera head 5119 in synchronization with the irradiation timing. According to this method, color images can be obtained without providing the image capturing element with a color filter.

Also, the driving of the light-source device 5157 may be controlled so that the intensity of output light is changed in an every predetermined period of time. An image having a high dynamic range without so-called crushed shadows and blown highlights can be generated by controlling the driving of the image capturing element of the camera head 5119 in synchronization with the timing of changing the intensity of the light to acquire images by time division and synthesizing the images.

Also, the light-source device 5157 may be structured to be able to supply the light having a predetermined wavelength band corresponding to special light observation. In the special light observation, for example, a so-called narrow-band-light observation (Narrow Band Imaging), in which images of predetermined tissues such as blood vessels of a mucus membrane surface layer are captured at a high contrast, is carried out by radiating the light having a narrower band compared with the irradiation light (in other words, white light) of a normal observation by using the wavelength dependency of light absorption of the body tissues. Alternatively, in the special light observation, a fluorescence observation of obtaining images by fluorescence generated by irradiation of excitation light may be carried out. As the fluorescence observation, for example, a fluorescence observation (autofluorescence observation) of irradiating body tissues with excitation light and observing the fluorescence from the body tissues or a fluorescence observation of locally injecting a reagent such as indocyanine green (ICG) into body tissues and irradiating the body tissues with the excitation light corresponding to the fluorescence wavelength of the reagent to obtain a fluorescent image can be carried out. The light-source device 5157 is structured to be able to supply narrow-band light and/or excitation light supporting such special light observations.

### 2. FIRST EMBODIMENT

«2.1. Configuration of System according to First Embodiment»

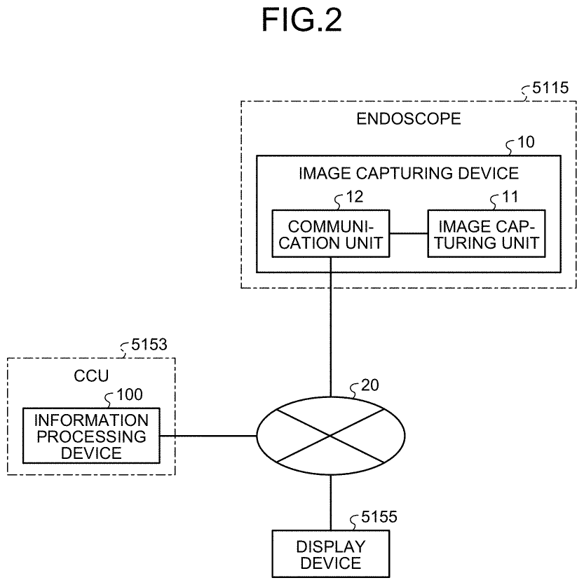

Next, a first embodiment of the present disclosure will be described in detail. FIG. 2 is a diagram illustrating a system configuration example according to a first embodiment of the present disclosure. As illustrated in FIG. 2, this system has an image capturing device 10, the display device 5155, and an information processing device 100. The image capturing device 10, the display device 5155, and the information processing device 100 are mutually connected via a network 20.

The image capturing device 10 is a device which captures in vivo images in a living body, which is an observation object. The image capturing device 10 may be, for example, the endoscope 5115 as described in FIG. 1. The image capturing device 10 has an image capturing unit 11 and a communication unit 12.

The image capturing unit 11 has a function to capture in vivo images in a living body, which is an observation object. The image capturing unit 11 according to the present example is structured to include, for example, an image capturing element such as a charge coupled device (CCD) or a complementary MOS (CMOS). The image capturing unit 11 captures in vivo images at a predetermined frame rate (FPS: Frames Per Second).

Herein, in vivo images according to the present embodiment widely include images (Biological Imaging) acquired from biology viewpoints for clinical, medical, and experimental uses, and image capturing objects are not limited to humans.

The communication unit 12 has a function to carry out information communication with the information processing device 100 via the network 20. For example, the communication unit 12 transmits the in vivo images, which have been captured by the image capturing unit 11, to the information processing device 100 in chronological order.

The information processing device 100 receives the in vivo images from the image capturing device 10 in chronological order. In the following descriptions, the in vivo images received from the image capturing device 10 will be described as “input images”. The information processing device 100 determines whether the input images include intraoperatively generated matters or not and generates output images based on the determination result and the input images. Examples of the substances generated in an operation are smoke and mist. The input images are also referred to as “medical images” and “intraoperative images”.

The information processing device 100 transmits the output images to the display device 5155. As described later, if the input images include smoke or mist, the information processing device 100 generates output images from which the smoke or the mist has been eliminated. The information processing device 100 may be, for example, the CCU 5153 as described in FIG. 1.

The display device 5155 receives the output images from the information processing device 100 and displays the received output images. The descriptions about the other display devices 5155 are similar to the descriptions about the display devices 5155 of FIG. 1.

«2.2. Configuration of Information Processing Device according to First Embodiment»

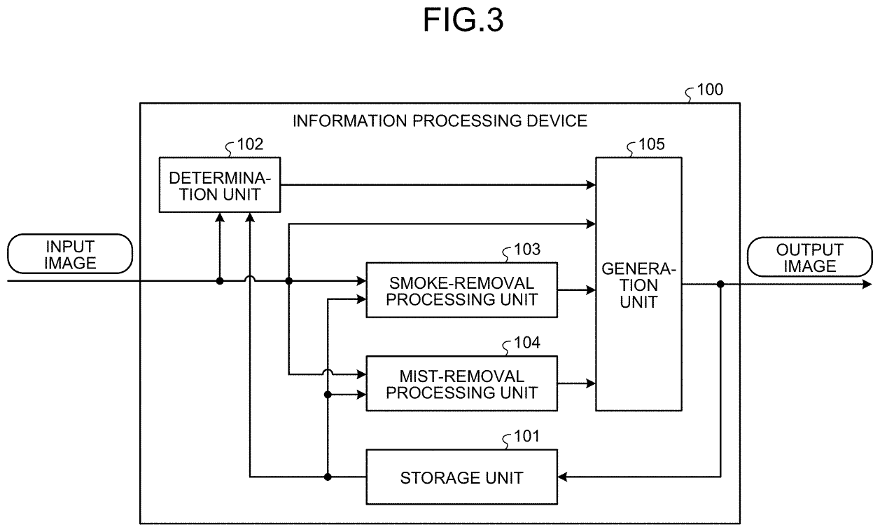

Next, the information processing device 100 according to the first embodiment of the present disclosure will be described in detail. FIG. 3 is a diagram illustrating a configuration example of the information processing device according to the first embodiment of the present disclosure. As illustrated in FIG. 3, the information processing device 100 has a storage unit 101, a determination unit 102, a smoke-removal processing unit 103, a mist-removal processing unit 104, and a generation unit 105.

Every time the input image is received from the image capturing device 10, the information processing device 100 inputs the input image to each of the determination unit 102, the smoke-removal processing unit 103, and the mist-removal processing unit 104. Although illustration is omitted in FIG. 3, it is assumed that the information processing device 100 has a communication unit which carries out information communication with the image capturing device 10 and the display device 5155 via the network 20.

((Storage Unit 101))

The storage unit 101 is a storage device which stores the information of the latest output image generated by the generation unit 105. The output image is an image in which smoke and mist has not been generated (or smoke and mist has been removed). The output image stored in the storage unit 101 is updated every time a new output image is output from the generation unit 105.

The storage unit 101 corresponds to a semiconductor memory element such as a random access memory (RAM), a read only memory (ROM), or a flash memory (Flash Memory) or a storage device such as a hard disk drive (HDD). The storage unit 101 may be either one of a volatile memory and a non-volatile memory or may use both of them.

((Determination Unit 102))

The determination unit 102 is a processing unit which determines whether the input image includes smoke or mist or not based on the input image. If it is determined that smoke or mist is included, the determination unit 102 determines the generation amounts of the smoke and the mist. Also, the determination unit 102 calculates the generation probabilities of the smoke and the mist. The generation probabilities of the smoke and the mist correspond to the ratio of the smoke to the mist.

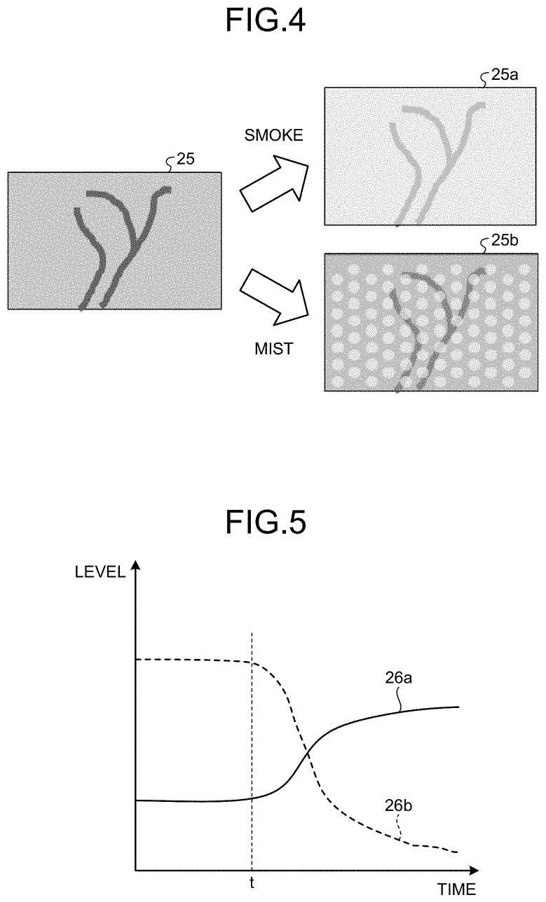

Herein, characteristics of each of smoke and mist will be described. FIG. 4 is a diagram for describing the characteristics of each of smoke and mist. In FIG. 4, an image 25 is an input image in which smoke and mist has not been generated. An image 25a is an input image in which smoke has been generated. An image 25b is an input image in which mist has been generated.

When smoke is generated, as illustrated in the image 25a, the smoke spreads comparatively uniformly like a fog. A smoked part has the characteristics that the entirety thereof becomes somewhat white and the background contrast thereof is lowered since light reflection occurs and transmittance is lowered.

When mist is generated, as illustrated in the image 25b, like smoke, the image becomes somewhat white, and the background contrast is lowered. Since mist is basically a gathering of water vapor or water droplets, the difference in the transmittance between parts with water droplets and parts without water droplets becomes large, and parts like an uneven pattern in which the background cannot be seen are formed.

Subsequently, the relation between generation of smoke and mist and brightness and color saturation will be described. FIG. 5 is a diagram illustrating the relation between generation of smoke and mist and brightness and color saturation. In a graph illustrated in FIG. 5, a horizontal axis is an axis corresponding to time. A vertical axis is an axis corresponding to a level (value) of brightness or color saturation. A line 26a represents the relation between brightness and time. A line 26b illustrates the relation between color saturation and time.

As illustrated in FIG. 5, when smoke (or mist) is generated at time t and the generation amount of the smoke (or mist) increases as time passes, the level of brightness increases and the level of color saturation is lowered along with increase in the generation amount.

An example of a process in which the determination unit 102 determines whether the input image includes smoke or mist or not will be described. The determination unit 102 calculates a reference value of brightness and a reference value of color saturation based on the output image stored in the storage unit 101. The determination unit 102 converts pixel values of the output image to brightness and color saturation. The determination unit 102 calculates the average value of the brightness of the output image as the reference value of brightness. The determination unit 102 calculates the average value of the color saturation of the output image as the reference value of color saturation.

When input of the input image is received, the determination unit 102 converts the pixel values, which are included in the input image, to brightness and color saturation. For example, if the average value of the brightness of the input image is less than the reference value of brightness and the average value of the color saturation of the input image is less than the reference value of the color saturation, the determination unit 102 determines that the input image includes smoke or mist.

When the determination unit 102 determines that the input image includes smoke or mist, the determination unit 102 executes a process of determining the generation amount of the smoke and the mist and a process of determining the generation probabilities of the smoke and the mist.

An example of the process in which the determination unit 102 determines the generation amounts of the smoke and the mist will be described. For example, the determination unit 102 divides the input image into plural blocks and calculates time changes in brightness and color saturation for each block.

For example, the determination unit 102 calculates the difference between the brightness of a block BOᵢⱼ of the output image and the brightness of a block BIᵢⱼ of the input image as a time change in brightness. BOᵢⱼ represents a block of an i-th row and a j-th column among the divided blocks of the output image. BIᵢⱼ represents a block of an i-th row and a j-th column among the divided blocks of the input image.

The determination unit 102 calculates the difference between the color saturation of the block BOᵢⱼ of the output image and the color saturation of the block BIᵢⱼ of the input image as a time change in color saturation.

The determination unit 102 compares each block of the output image with each block of the input image to calculate the time changes in brightness and color saturation for each block. The determination unit 102 determines the block(s), in which the time changes in brightness and color saturation are equal to or higher than threshold values, as the blocks in which smoke or mist has been generated among the blocks of the input image.

For example, the determination unit 102 specifies the generation amounts of the smoke and the mist based on a “generation-amount specifying table (illustration omitted)”, which defines the relation between the rate of smoke or mist generated blocks to all the blocks of the input image, time changes in brightness and color saturation, and the generation amounts of smoke and mist. It is assumed that the information of the generation-amount specifying table is set in advance.

Subsequently, an example of a process in which the determination unit 102 determines the generation probabilities (ratio) of the smoke and the mist will be described. The determination unit 102 calculates a dynamic range for each block of the input image. For example, the determination unit 102 scans the brightness (or pixel values) included in one block, specifies a maximum value of the brightness and a minimum value of the brightness, and calculates the difference between the maximum value of the brightness and the minimum value of the brightness as the dynamic range.

The determination unit 102 determines the generation probabilities of smoke and mist with respect to one block based on a “generation-ratio specifying table (illustration omitted)” in which the relation between dynamic ranges and the generation probabilities of smoke and mist are defined. For example, since there is a tendency that local contrasts of an image are increased more by mist when compared with smoke, the larger the dynamic range, the higher the generation probability of mist compared with the generation probability of smoke.

The determination unit 102 executes the process of calculating the generation probabilities of smoke and mist for each block in which smoke or mist has been generated. The determination unit 102 specifies representative values of the generation probabilities of smoke and mist based on the calculated generation probabilities of smoke and mist. For example, the determination unit 102 specifies the average values, median values, or the like of the generation probabilities of smoke and mist as the representative values of the generation probabilities of the smoke and mist.

For example, the determination unit 102 adjusts a generation probability P1 of smoke and a generation probability P2 of mist so that the generation probability which is the total of the generation probability P1 of smoke and the generation probability P2 of mist becomes 100%.

The determination unit 102 outputs the determination result to the generation unit 105. The determination result includes the information on whether smoke or mist has been generated or not in the input image. If smoke or mist has been generated, the determination result further includes the generation amounts of the smoke and the mist and the representative values of the generation probabilities of the smoke and the mist. In the following description, the representative values of the generation probabilities of the smoke and the mist will be simply described as the generation probabilities of the smoke and the mist.

((Smoke-removal Processing Unit 103))

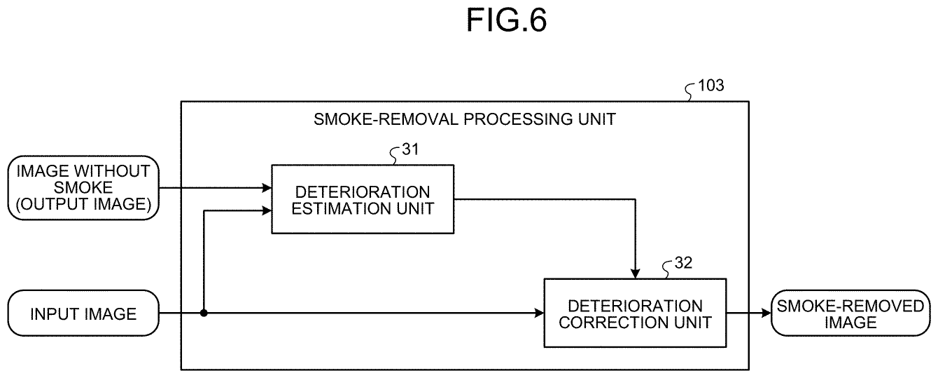

The smoke-removal processing unit 103 is a processing unit which generates a smoke-removed image which is the input image from which the smoke has been reduced or removed. In the following description, “reducing or removing the smoke from the input image” will be appropriately described as “removing the smoke from the input image”. FIG. 6 is a diagram illustrating a configuration example of a smoke-removal processing unit according to the first embodiment of the present disclosure. As illustrated in FIG. 6, the smoke-removal processing unit 103 has a deterioration estimation unit 31 and a deterioration correction unit 32.

The deterioration estimation unit 31 is a processing unit which estimates deterioration of the input image based on the input image and the output image. The deterioration estimation unit 31 outputs a deterioration estimation result to the deterioration correction unit 32. For example, the deterioration estimation unit 31 executes a histogram converting process and/or a correction-amount-map calculating process. Hereinafter, the histogram converting process and/or the correction-amount-map calculating process executed by the deterioration estimation unit 31 will be described.

An example of the histogram converting process executed by the deterioration estimation unit 31 will be described. The deterioration estimation unit 31 carries out conversion so that a histogram h_{S}(sⱼ) of the input image matches a histogram h_{T}(tⱼ) of the output image (target image). Herein, sⱼ represents a j-th pixel value of the input image. A j-th pixel value of the output image is represented by tⱼ. For example, the pixel values of the input image and the output image have values of 0 to 255.

The deterioration estimation unit 31 normalizes a histogram by the number of pixels and obtains a probability density function. For example, a probability density function p_{S}(sⱼ) of the input image is defined by Equation (1). The deterioration estimation unit 31 calculates the probability density function p_{S}(sⱼ) of the input image based on Equation (1).

$${p}_{s} ( {s}_{j} ) = \frac{{h}_{s} ( {s}_{j} )}{{\sum }_{j} {h}_{s} ( {s}_{j} )} \qquad ( 1 )$$

A probability density function p_{T}(tⱼ) of the output image is defined by Equation (2). The deterioration estimation unit 31 calculates the probability density function p_{T}(tⱼ) based on Equation (2).

$${p}_{T} ( {t}_{j} ) = \frac{{h}_{T} ( {t}_{j} )}{{\sum }_{j} {h}_{T} ( {t}_{j} )} \qquad ( 2 )$$

The deterioration estimation unit 31 obtains the probability density function and then obtains a cumulative distribution function of the probability density function. For example, a cumulative distribution function F_{S}(sₖ) of the input image is defined by Equation (3). The deterioration estimation unit 31 calculates the cumulative distribution function F_{S}(sₖ) based on Equation (3). If the pixel values of the input image have values of 0 to 255, k=255.

$${F}_{S} ( {s}_{k} ) = {\sum }_{j = 0}^{k} {p}_{S} ( {s}_{j} ) \qquad ( 3 )$$

A cumulative distribution function F_{T}(tₖ) of the output image is defined by Equation (4). The deterioration estimation unit 31 calculates the cumulative distribution function F_{T}(tₖ) based on Equation (4). If the pixel values of the output image have values of 0 to 255, k=255.

$${F}_{T} ( {t}_{k} ) = {\sum }_{j = 0}^{k} {p}_{T} ( {t}_{j} ) \qquad ( 4 )$$

The deterioration estimation unit 31 calculates an inverse function F_{T}⁻¹(sₖ) of F_{T}(sₖ) and converts the pixel values of the input image so that F_{S}(sₖ)=F_{T}(sₖ) is satisfied. For example, the deterioration estimation unit 31 generates a histogram conversion image H(I) by converting each of the pixel values sⱼ (j=0 to 255) of the input image to an output pixel value oⱼ based on Equation (5).

Oⱼ=F_{T}⁻[F_{S}(sⱼ)] (5)

Herein, the deterioration estimation unit 31 is not necessarily required to carry out the above described histogram converting process with respect to the entire image, but may carry out the process in a particular region or a grid unit of the image.

An example of the correction-amount-map calculating process executed by the deterioration estimation unit 31 will be described. The deterioration estimation unit 31 calculates a correction-amount map M based on Equation (6). As shown in Equation (6), the deterioration estimation unit 31 calculates the correction-amount map M of contrast by calculating the difference between a histogram conversion image (I) and an input image I.

M=H(I)−I (6)

Also, the deterioration estimation unit 31 generates a shaped correction-amount map F(I,M) by shaping a correction-amount map of contrast by a guided filter using the input image as a guide image. The shaped correction-amount map F(I,M) is a map which is the correction-amount map M shaped in accordance with an edge of the input image I. In this manner, by using the shaped correction-amount map F(I,M), image deterioration around the edge which can occur when the positions of the correction-amount map M and the input image I are misaligned can be prevented.

Note that the deterioration estimation unit 31 may generate the shaped correction-amount map F(I,M) by using a guided filter described in any of Non-Patent Literatures 1, 2, and 3 shown below.

Non-Patent Literature 1: Kopf, Johannes, et al. “Joint bilateral upsampling.” ACM Transactions on Graphics (ToG). Vol. 26. No. 3. ACM, 2007.

Non-Patent Literature 2: He, Kaiming, Jian Sun, and Xiaoou Tang. “Guided image filtering.” European conference on computer vision. Springer, Berlin, Heidelberg, 2010.

Non-Patent Literature 3: Gastal, Eduardo S L, and Manuel M. Oliveira. “Domain transform for edge-aware image and video processing. “ACM Transactions on Graphics (ToG). Vol. 30. No. 4. ACM, 2011.

The deterioration estimation unit 31 outputs the shaped correction-amount map F(I,M) to the deterioration correction unit 32 as a deterioration estimation result. In the shaped correction-amount map F(I,M), pixel values of pixels are defined.

The deterioration correction unit 32 is a processing unit which generates a smoke-removed image by correcting the input image based on the deterioration estimation result. For example, the deterioration correction unit 32 generates a smoke-removed image OA based on Equation (7). Equation (7) means executing, with respect to each pixel, a process of adding the pixel values of pixels of the input image I to the pixel values of pixels at the same positions in the shaped correction-amount map F(I,M). The deterioration correction unit 32 outputs the information of the smoke-removed image to the generation unit 105.

O_{A}=F(I,M)+I (7)

((Mist-Removal Processing Unit 104))

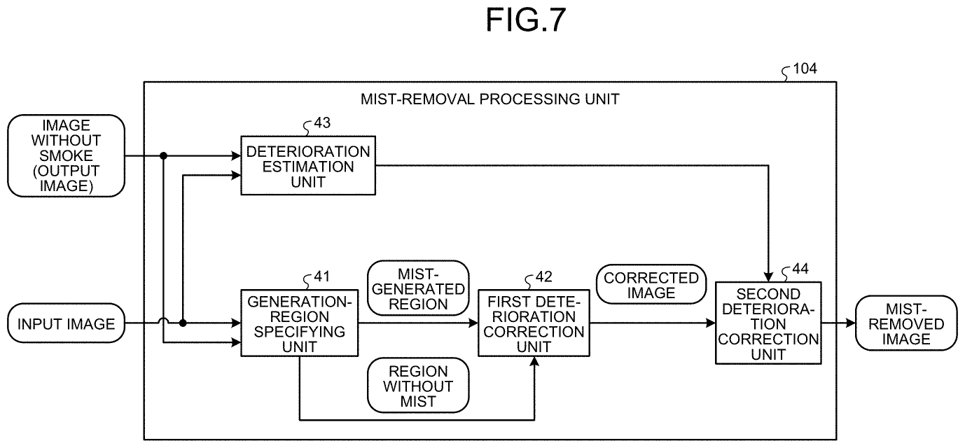

The mist-removal processing unit 104 is a processing unit which generates a mist-removed image which is the input image from which mist has been reduced or removed. In the following description, “reducing or removing the mist from the input image” will be appropriately described as “removing the mist from the input image”. FIG. 7 is a diagram illustrating a configuration example of a mist-removal processing unit according to the first embodiment of the present disclosure. As illustrated in FIG. 7, the mist-removal processing unit 104 has a generation-region specifying unit 41, a first deterioration correction unit 42, a deterioration estimation unit 43, and a second deterioration correction unit 44.

The generation-region specifying unit 41 is a processing unit which compares the input image with the output image and determines a mist-generated regions in the regions of the input image. For example, as well as the determination unit 102, the generation-region specifying unit 41 determines the block(s), in which the time changes in brightness and color saturation are equal to or higher than threshold values, as the blocks in which smoke or mist has been generated among the blocks of the input image. Also, the generation-region specifying unit 41 specifies the blocks in which brightness is equal to or higher than a threshold value Thy as mist-generated regions among the blocks in which smoke or mist has been generated.

Note that the generation-region specifying unit 41 may specify mist-generated regions by other processes. For example, the generation-region specifying unit 41 may divide the input image into plural blocks and specify the blocks in which brightness is equal to or higher than the threshold value Thy as mist-generated regions among the plural blocks.

The generation-region specifying unit 41 outputs the information of the mist-generated regions and the information of the regions in which mist has not been generated to the first deterioration correction unit 42. Also, the generation-region specifying unit 41 outputs the information of the input image to the first deterioration correction unit 42.

The first deterioration correction unit 42 is a processing unit which corrects the mist-generated regions in the input image according to the information of the regions in which mist has not been generated. The first deterioration correction unit 42 divides the input image into plural blocks and sorts the divided plural blocks into mist-generated blocks and the blocks in which mist has not been generated. In the following descriptions, the block in which mist has been generated is described as a first block. The block in which mist has not been generated is described as a second block.

The first deterioration correction unit 42 selects a first block and selects a second block which is positioned within a predetermined distance from the selected first block. The first deterioration correction unit 42 adjusts the contrast of the selected first block so that the contrast becomes the same as the contrast of the selected second block. If plural second blocks which are positioned within a predetermined distance from the selected first block are present, the first deterioration correction unit 42 may calculate the average value of the contrasts of the plural second blocks and adjust the contrast of the first block so that the contrast becomes the same as the average value of the contrasts of the plural second blocks.

The first deterioration correction unit 42 corrects the input image by repeatedly executing the above process with respect to each first block included in the input image.

The first deterioration correction unit 42 outputs the corrected input image to the second deterioration correction unit 44. In the following descriptions, the corrected input image will be described as “corrected image”.

The deterioration estimation unit 43 is a processing unit which estimates deterioration of the input image based on the input image and the output image. The deterioration estimation unit 43 outputs a deterioration estimation result to the second deterioration correction unit 44. The process of the deterioration estimation unit 43 is similar to the process of the deterioration estimation unit 31. For example, the deterioration estimation result output from the deterioration estimation unit 43 to the second deterioration correction unit 44 is a shaped correction-amount map F(I,M).

The second deterioration correction unit 44 is a processing unit which generates a mist-removed image by correcting the corrected image based on the deterioration estimation result. For example, the second deterioration correction unit 44 generates a mist-removed image O_{B} based on Equation (8). Equation (8) means executing, with respect to each pixel, a process of adding the pixel values of pixels of the corrected image I_{B} to the pixel values of pixels at the same positions in the shaped correction-amount map F(I,M). The second deterioration correction unit 44 outputs the information of the mist-removed image to the generation unit 105.

O_{B}=F(I,M)+I_{B} (8)

((Generation Unit 105))

The generation unit 105 is a processing unit which

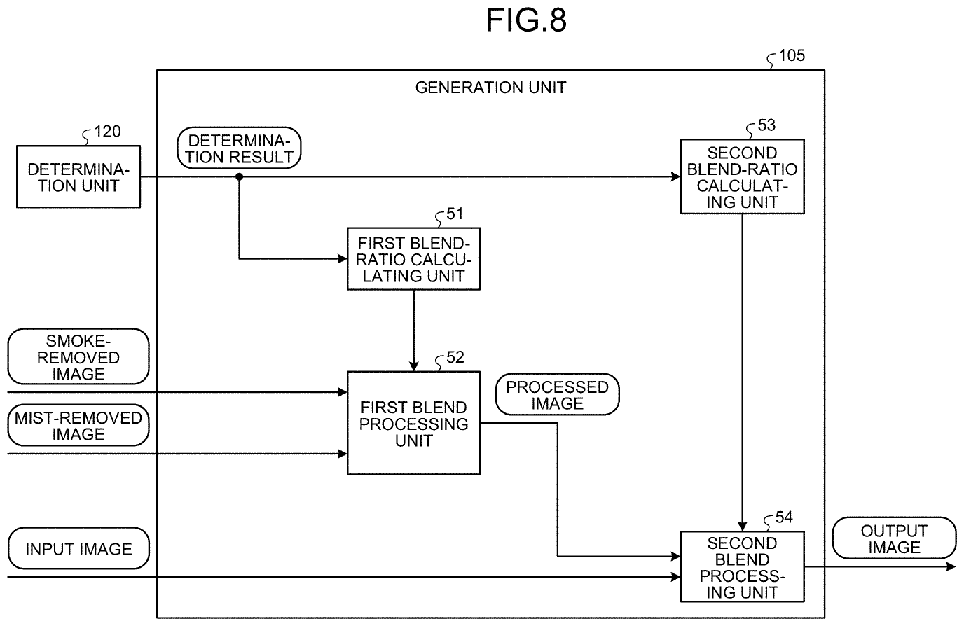

generates the output image based on the determination result from the determination unit 102 and the input image. The generation unit 105 outputs the information of the output image to the display device 5155. The influence of smoke or mist is eliminated from the entirety of the output image. FIG. 8 is a diagram illustrating a configuration example of the generation unit according to the first embodiment of the present disclosure. As illustrated in FIG. 8, the generation unit 105 has a first blend-ratio calculating unit 51, a first blend processing unit 52, a second blend-ratio calculating unit 53, and a second blend processing unit 54.

The first blend-ratio calculating unit 51 is a processing unit which calculates a blend ratio α of the smoke-removed image and the mist-removed image based on the determination result of the determination unit 102. The first blend-ratio calculating unit 51 sets the generation probabilities of smoke and mist as the blend ratio α. For example, if an adjustment is made that the total of the generation probability P1 of the smoke and the generation probability P2 of the mist becomes 100%, the blend ratio α of the smoke and the mist becomes P1:P2. The first blend-ratio calculating unit 51 outputs the information of the blend ratio α to the first blend processing unit 52.

The first blend processing unit 52 is a processing unit which generates a processed image by blending (synthesizing) the smoke-removed image and the mist-removed image based on the blend ratio α. The first blend processing unit 52 outputs the information of the processed image to the second blend processing unit 54.

For example, the pixel value of a pixel in an i-th row and a j-th column of the processed image is S3ᵢⱼ, the pixel value of a pixel in an i-th row and a j-th column of the smoke-removed image is S1ᵢⱼ, and the pixel value of a pixel in an i-th row and a j-th column of the mist-removed image is S2ᵢⱼ. In this case, the first blend processing unit 52 calculates the pixel value S3ᵢⱼ of the processed pixel by Equation (9).

S1ᵢⱼ=P1/(P1+P2)×S1ᵢⱼ+P2/(P1+P2)×S2ᵢⱼ (9)

The first blend processing unit 52 generates the processed image by calculating the pixel values of the pixels of the processed image based on Equation (9).

The second blend-ratio calculating unit 53 is a processing unit which calculates a blend ratio β of the processed image and the input image based on the determination result of the determination unit 102. For example, the second blend-ratio calculating unit 53 calculates the blend ratio β based on a “blend-ratio specifying table (illustration omitted)”, which defines the relation between the generation amounts of smoke and mist and the blend ratio β.

For example, descriptions will be given on an assumption that the blend ratio β of the processed image and the input image is P3:P4. For example, the blend-ratio specifying table is set so that the higher the generation amounts of the smoke and mist, the higher the ratio P3 of the processed image than the ratio of the input image P4. The second blend-ratio calculating unit 53 outputs the information of the blend ratio β to the second blend processing unit 54.

The second blend processing unit 54 is a processing unit which generates the output image by blending (synthesizing) the processed image and the input image based on the blend ratio β. For example, the pixel value of a pixel in an i-th row and a j-th column of the processed image is S3ᵢⱼ, the pixel value of a pixel in an i-th row and a j-th column of the input image is S4ᵢⱼ, and the pixel value of a pixel in an i-th row and a j-th column of the output image is S5ᵢⱼ. In this case, the second blend processing unit 54 calculates the pixel value S5ᵢⱼ of the output image by Equation (10).

S5ᵢⱼ=P3/(P3+P4)×S3ᵢⱼ+P4/(P3+P4)×S4ᵢⱼ (10)

The second blend processing unit 54 generates the output image by calculating each of the pixel values of the pixels of the processed image based on Equation (10).

«2.3. Flow of Actions of Information Processing Device 100»

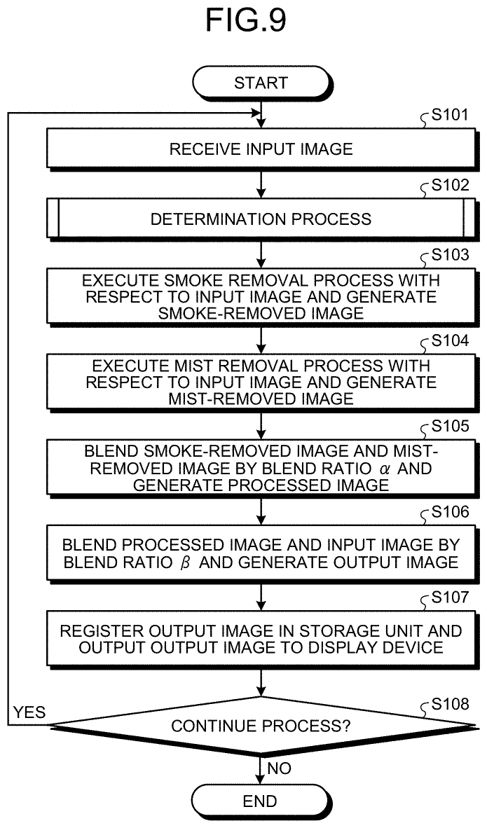

Next, a flow of actions of the information processing device 100 according to the first embodiment of the present disclosure will be described. FIG. 9 is a flow chart illustrating a flow of basic actions of the information processing device 100 according to the first embodiment of the present disclosure.

In FIG. 9, the information processing device 100 receives the input image from the image capturing device 10 (step S101). The determination unit 102 of the information processing device 100 executes a determination process (step S102).

The smoke-removal processing unit 103 of the information processing device 100 executes a smoke removal process with respect to the input image and generates a smoke-removed image (step S103). The mist-removal processing unit 104 of the information processing device 100 executes a mist removal process with respect to the input image and generates a mist-removed image (step S104).

The generation unit 105 of the information processing device 100 blends the smoke-removed image and the mist-removed image by the blend ratio α and generates the processed image (step S105). The generation unit 105 blends the processed image and the input image by the blend ratio β and generates the output image (step S106).

The generation unit 105 registers the output image in the storage unit 101 and outputs the output image to the display device 5155 (step S107). If the process is to be continued (step S108, Yes), the information processing device 100 transitions to step S101. On the other hand, if the process is not to be continued (step S108, No), the information processing device 100 terminates the process.

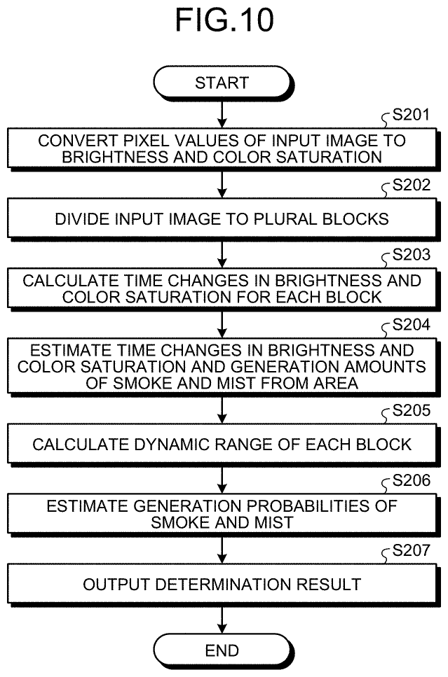

Next, a flow of actions of the determination process shown in step S102 of FIG. 9 will be described. FIG. 10 is a flow chart illustrating the flow of the actions of the determination unit according to the first embodiment of the present disclosure.

The determination unit 102 of the information processing device 100 converts the pixel values of the input image to brightness and color saturation (step S201). The determination unit 102 divides the input image into plural blocks (step S202).

The determination unit 102 calculates time changes in the brightness and the color saturation for each block (step S203). The determination unit 102 estimates the generation amounts of smoke and mist from the time changes and areas of the brightness and the color saturation (step S204).

The determination unit 102 calculates the dynamic range of each block (step S205). The determination unit 102 estimates the generation probabilities of the smoke and mist (step S206). The determination unit 102 outputs the determination result to the generation unit 105 (step S207).

«2.4. Effects of Information Processing Device according to First Embodiment»

According to the information processing device 100 according to the first embodiment of the present disclosure, whether the input image includes smoke or mist or not is determined based on the input image, and the output image from which the smoke or mist has been eliminated is generated based on the determination result and the input image. By virtue of this, even if smoke or mist is generated during an operation of an endoscope operation and deteriorates view, clearer view can be ensured by using the information processing device 100 regardless of the amounts of generated smoke or mist.

According to the information processing device 100, the smoke-removed image is generated by adjusting the contrast of the input image by the smoke-removal processing unit 103 so that the contrast becomes the same as the contrast of the output image in which smoke and mist has not been generated. By virtue of this, the smoke included in the input image can be appropriately eliminated.

According to the information processing device 100, the mist-removal processing unit 104 adjusts the input image so that the contrast of the mist-generated region becomes the same as the contrast of the region in which mist has not been generated and then further adjusts the contrast of the input image so that the contrast becomes the same as the contrast of the output image in which smoke and mist has not been generated. By adjusting the contrast of the input image in two levels in this manner, mist having an uneven pattern which has different characteristics from smoke can be appropriately eliminated from the input image.

According to the information processing device 100, the generation probabilities of smoke and mist are determined based on the input image. By virtue of this, the smoke-removed image and the mist-removed image can be synthesized by the blend ratio α, which is based on the generation probabilities of smoke and mist, and the smoke and the mist included in the input image can be appropriately eliminated. According to the information processing device 100, the generation amount of smoke or mist is determined based on the input image. By virtue of this, the processed image and the input image can be synthesized by the blend ratio β, which is based on the generation amount of the smoke or mist, and the smoke and the mist included in the input image can be appropriately eliminated.

Also, according to the information processing device 100, since the process about removal of the smoke and the mist is executed with respect to the input image, the effect of reducing the smoke or mist can be immediately obtained when the smoke or mist is generated.

### 3. SECOND EMBODIMENT

«3.1. Configuration of System according to Second Embodiment»

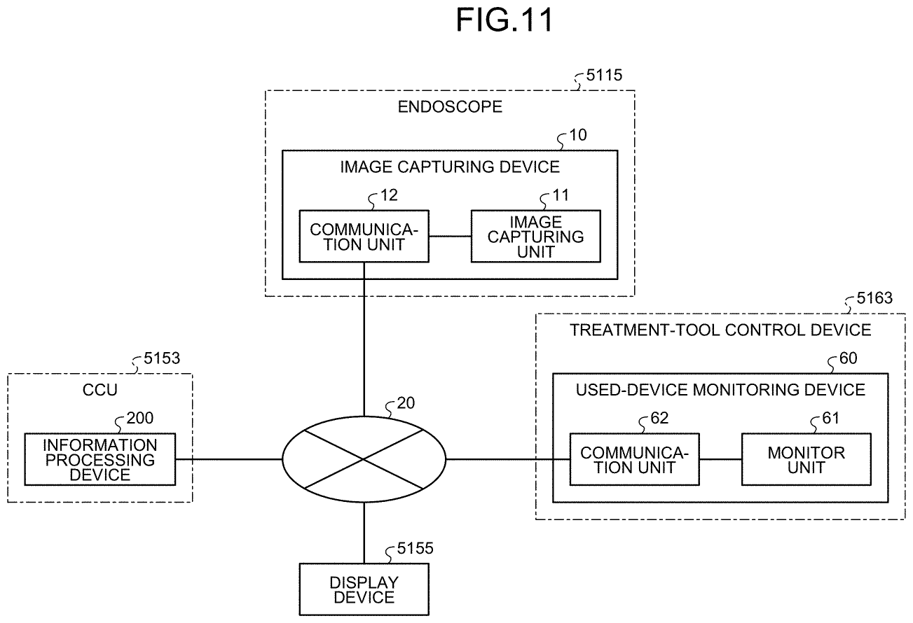

Next, a second embodiment of the present disclosure will be described in detail. FIG. 11 is a diagram illustrating a system configuration example according to the second embodiment of the present disclosure. As illustrated in FIG. 11, this system has the image capturing device 10, a used-device monitoring device 60, the display device 5155, and an information processing device 200. The image capturing device 10, the used-device monitoring device 60, the display device 5155, and the information processing device 200 are mutually connected via the network 20.

The descriptions about the image capturing device 10 and the display device 5155 are similar to the descriptions about the image capturing device 10 and the display device 5155 described in FIG. 2.

The used-device monitoring device 60 is a device which is connected to an electric scalpel, an ultrasonic clotting/incising device, and/or the like, which are omitted in illustration, and monitors whether the electric scalpel and/or the ultrasonic clotting/incising device is being used or not. The used-device monitoring device 60 may be, for example, the treatment-tool control device 5163 described in FIG. 1. The used-device monitoring device 60 has a monitor unit 61 and a communication unit 62.

The monitor unit 61 is a processing unit which monitors the usage status of the electric scalpel and/or the ultrasonic clotting/incising device. For example, when a control signal of usage start is received from a usage start button of the electric scalpel or the ultrasonic clotting/incising device, the monitor unit 61 determines that the electric scalpel or the ultrasonic clotting/incising device is in use. The monitor unit 61 generates used device information. The used device information includes the information whether the electric scalpel is in use or not and the information whether the ultrasonic clotting/incising device is in use or not.

For example, the electric scalpel stops bleeding or carries out incision with respect to an affected part of the patient 5185 by the heat generated by a high-frequency current. The electric scalpel burns a treatment part and therefore has a characteristic that smoke is easily generated.

The ultrasonic clotting/incising device carries out clotting or incision of an affected part of the patient 5185 by friction caused by ultrasonic oscillations. The ultrasonic clotting/incising device has a characteristic that mist is easily generated by the ultrasonic oscillations.

The communication unit 62 has a function to carry out information communication with the information processing device 200 via the network 20. For example, the communication unit 62 transmits the used device information, which has been generated by the monitor unit 61, to the information processing device 200.

«3.2. Configuration of Information Processing Device according to Second Embodiment»

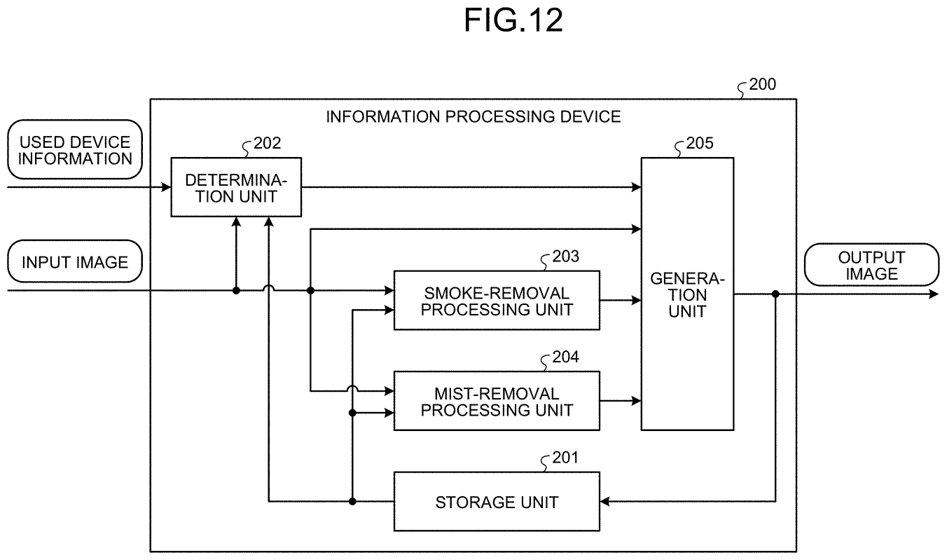

Next, the information processing device 200 according to the second embodiment of the present disclosure will be described in detail. FIG. 12 is a diagram illustrating a configuration example of the information processing device according to the second embodiment of the present disclosure. As illustrated in FIG. 12, the information processing device 200 has a storage unit 201, a determination unit 202, a smoke-removal processing unit 203, a mist-removal processing unit 204, and a generation unit 205.

Every time the input image is received from the image capturing device 10, the information processing device 200 inputs the input image to each of the determination unit 202, the smoke-removal processing unit 203, and the mist-removal processing unit 204. Every time the used device information is received from the used-device monitoring device 60, the information processing device 200 inputs the used device information to the determination unit 202. Although illustration is omitted in FIG. 12, it is assumed that the information processing device 200 has a communication unit which carries out information communication with the image capturing device 10, the used-device monitoring device 60, and the display device 5155 via the network 20.

((Storage Unit 201))

The storage unit 201 is a storage device which stores the information of the latest output image generated by the generation unit 205. The output image is an image in which smoke and mist has not been generated (or smoke and mist has been removed). The output image stored in the storage unit 201 is updated every time a new output image is output from the generation unit 205.

The storage unit 201 corresponds to a semiconductor memory element such as a RAM, a ROM, or a flash memory or a storage device such as a HDD. The storage unit 201 may be either one of a volatile memory and a non-volatile memory or may use both of them.

((Determination Unit 202))

The determination unit 202 is a processing unit which determines whether the input image includes smoke or mist or not based on the used device information and the input image. If it is determined that smoke or mist is included, the determination unit 202 determines the generation amounts of the smoke and the mist. Also, the determination unit 202 calculates the generation probabilities of the smoke and the mist. The generation probabilities of the smoke and the mist correspond to the ratio of the smoke to the mist.

An example of a process in which the determination unit 202 determines whether the input image includes smoke or mist or not will be described. For example, if the used device information includes the information that the electric scalpel or the ultrasonic clotting/incising device is used, the determination unit 202 determines that the input image includes smoke or mist.

Note that, in the cases that the input image has been determined to include smoke or mist based on the input image and that the used device information includes the information that the electric scalpel or the ultrasonic clotting/incising device is used, the determination unit 202 may determine that the input image includes smoke or mist. The process that the determination unit 202 determines that the input image includes smoke or mist based on the input image is similar to the process of the determination unit 102 of the first embodiment.

When the determination unit 202 determines that the input image includes smoke or mist, the determination unit 202 executes a process of determining the generation amount of the smoke and the mist and a process of determining the generation probabilities of the smoke and the mist.

The process in which the determination unit 202 determines the generation amounts of the smoke and the mist is similar to the process of the determination unit 102 of the first embodiment.

An example of a process in which the determination unit 202 determines the generation probabilities (ratio) of the smoke and the mist will be described. As well as the determination unit 102 of the first embodiment, the determination unit 202 calculates the generation probability P1 of the smoke and the generation probability P2 of the mist (representative values of the generation probabilities of the smoke and the mist) based on the dynamic range.

Herein, the determination unit 202 corrects the generation probability P1 of the smoke and the generation probability P2 of the mist based on the used device information. If the electric scalpel is in use and the ultrasonic clotting/incising device is not in use according to the used device information, the determination unit 202 updates the generation probability P1 of the smoke by adding a predetermined probability value to the generation probability P1 of the smoke. Also, the determination unit 202 updates the generation probability P2 of the mist by subtracting a predetermined probability value from the generation probability P2 of the mist.

If the electric scalpel is not in use and the ultrasonic clotting/incising device is in use according to the used device information, the determination unit 202 updates the generation probability P1 of the smoke by subtracting a predetermined probability value from the generation probability P1 of the smoke. Also, the determination unit 202 updates the generation probability P2 of the mist by adding a predetermined probability value to the generation probability P2 of the mist.

If the electric scalpel and the ultrasonic clotting/incising device are in use based on the used device information, the determination unit 202 uses the generation probability P1 of the smoke and the generation probability P2 of the mist as generation probabilities without change.

The determination unit 202 outputs the determination result to the generation unit 205. The determination result includes the information on whether smoke or mist has been generated or not in the input image. If smoke or mist has been generated, the determination result further includes the generation amounts of the smoke and the mist and the generation probabilities P1 and P2 of the smoke and the mist.

((Smoke-Removal Processing Unit 203))

The smoke-removal processing unit 203 is a processing unit which generates a smoke-removed image which is the input image from which the smoke has been removed. The smoke-removal processing unit 203 outputs the smoke-removed image to the generation unit 205. The descriptions about the smoke-removal processing unit 203 are similar to the process of the smoke-removal processing unit 103 of the first embodiment.

((Mist-Removal Processing Unit 204))

The mist-removal processing unit 204 is a processing unit which generates a mist-removed image which is the input image from which mist has been removed. The mist-removal processing unit 204 outputs the mist-removed image to the generation unit 205. The descriptions about the mist-removal processing unit 204 are similar to the process of the mist-removal processing unit 104 of the first embodiment.

((Generation Unit 205))

The generation unit 205 is a processing unit which generates the output image based on the determination result from the determination unit 202 and the input image. The generation unit 205 outputs the information of the output image to the display device 5155. The descriptions about the generation unit 205 are similar to the process of the generation unit 105 of the first embodiment.

«3.3. Effects of Information Processing Device according to Second Embodiment»

According to the information processing device 200 according to the second embodiment of the present disclosure, whether the input image includes smoke or mist or not is determined based on the used device information and the input information, and the generation probabilities of the smoke and the mist are corrected based on the used device information. By virtue of this, the determination precision on whether the input image includes smoke or mist or not can be improved. Also, by correcting the generation probabilities of the smoke and the mist based on the used device information, the smoke-eliminated image and the mist-eliminated image can be blended by a more appropriate blend ratio α.

### 4. THIRD EMBODIMENT

«4.1. Configuration of System according to Third Embodiment»

Next, a third embodiment of the present disclosure will be described in detail. A system configuration according to the third embodiment of the present disclosure is similar to the system configuration according to the second embodiment of the present disclosure described in FIG. 11. In the following descriptions, an information processing device according to the third embodiment will be described as an information processing device 300. Although illustration is omitted, the image capturing device 10, the used-device monitoring device 60, the display device 5155, and the information processing device 300 are mutually connected via the network 20.

«4.2. Configuration of Information Processing Device according to Third Embodiment»

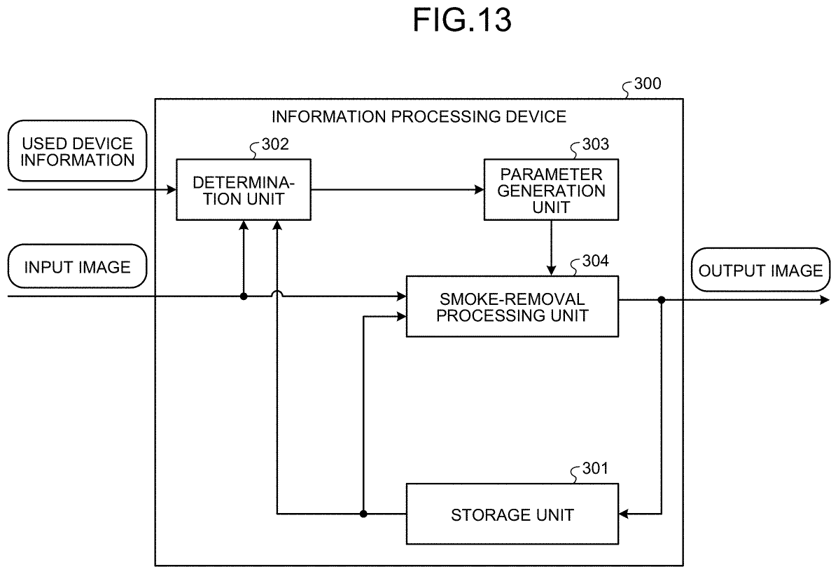

Next, the information processing device 300 according to the third embodiment of the present disclosure will be described in detail. FIG. 13 is a diagram illustrating a configuration example of the information processing device according to the third embodiment of the present disclosure. As illustrated in FIG. 13, the information processing device 300 has a storage unit 301, a determination unit 302, a parameter generation unit 303, and a smoke-removal processing unit 304.

Every time the input image is received from the image capturing device 10, the information processing device 300 inputs the input image to each of the determination unit 302 and the smoke-removal processing unit 203. Every time the used device information is received from the used-device monitoring device 60, the information processing device 300 inputs the used device information to the determination unit 302. Although illustration is omitted in FIG. 13, it is assumed that the information processing device 300 has a communication unit which carries out information communication with the image capturing device 10, the used-device monitoring device 60, and the display device 5155 via the network 20.

Note that the information processing device 300 is assumed to treat smoke or mist as smoke without discriminating between smoke and mist.

((Storage Unit 301))

The storage unit 301 is a storage device which stores the information of the latest output image generated by the smoke-removal processing unit 304. The output image is an image in which smoke has not been generated (or smoke has been removed). The output image stored in the storage unit 301 is updated every time a new output image is output from the smoke-removal processing unit 304.

The storage unit 301 corresponds to a semiconductor memory element such as a RAM, a ROM, or a flash memory or a storage device such as a HDD. The storage unit 301 may be either one of a volatile memory and a non-volatile memory or may use both of them.

((Determination Unit 302))

The determination unit 302 is a processing unit which determines whether the input image includes smoke or not based on the used device information and the input image. If it is determined that smoke is included, the determination unit 302 determines the generation amount of the smoke.

An example of a process in which the determination unit 302 determines whether the input image includes smoke or not will be described. For example, if the used device information includes the information that the electric scalpel or the ultrasonic clotting/incising device is used, the determination unit 302 determines that the input image includes smoke.

Note that, in the cases that the input image has been determined to include smoke based on the input image and that the used device information includes the information that the electric scalpel or the ultrasonic clotting/incising device is used, the determination unit 302 may determine that the input image includes smoke. The process that the determination unit 202 determines that the input image includes smoke (smoke or mist) based on the input image is similar to the process of the determination unit 102 of the first embodiment.

When the determination unit 302 determines that the input image includes smoke, the determination unit 302 executes a process of determining the generation amount of the smoke and a process of specifying a smoke generated region.

For example, the determination unit 302 divides the input image into plural blocks and calculates time changes in brightness and color saturation for each block.

The determination unit 302 calculates the difference between the brightness of a block BOᵢⱼ of the output image and the brightness of a block BIᵢⱼ of the input image as a time change in brightness. BOᵢⱼ represents a block of an i-th row and a j-th column among the divided blocks of the output image. BIᵢⱼ represents a block of an i-th row and a j-th column among the divided blocks of the input image.

The determination unit 302 calculates the difference between the color saturation of the block BOᵢⱼ of the output image and the color saturation of the block BIᵢⱼ of the input image as a time change in color saturation.

The determination unit 302 compares each block of the output image with each block of the input image to calculate the time changes in brightness and color saturation for each block.

The determination unit 102 specifies the block(s), in which the time changes in brightness and color saturation are equal to or higher than threshold values, as a smoke generated region among the blocks of the input image.

The determination unit 302 specifies the generation amounts of the smoke based on a “generation-amount specifying table (illustration omitted)”, which defines the relation between the rate of blocks of smoke generated regions to all the blocks of the input image, time changes in brightness and color saturation, and the generation amounts of smoke. It is assumed that the information of the generation-amount specifying table is set in advance.

The determination unit 302 outputs the determination result to the parameter generation unit 303. The determination result includes the information on whether smoke has been generated in the input image or not. If the smoke has been generated, the determination result further includes the information on the generation amount of the smoke and the generated region of the smoke.

((Parameter Generation Unit 303))

The parameter generation unit 303 is a processing unit which generates parameters of a smoke removal process based on the determination result of the determination unit 302. The parameters generated by the parameter generation unit 303 includes the information of on/off timing of the smoke removal process, the intensity level of the smoke removal process, and a region serving as a target of the smoke removal process.

An example of a process of the parameter generation unit 303 while the determination result includes the information that smoke is not generated in the input image will be described. The parameter generation unit 303 acquires the determination result of the determination unit 302 and, while the determination result includes the information that smoke is not generated in the input image, the parameter generation unit 303 generates a parameter setting the smoke removal process as “off” and outputs the parameter to the smoke-removal processing unit 304.

Note that, while the information that smoke has not been generated in the input image is included, the parameter generation unit 303 does not set, as parameters, the information of the intensity level of the smoke removal process and the region serving as a target of the smoke removal process.

Subsequently, an example of a process of the parameter generation unit 303 while the determination result includes the information that smoke is generated in the input image will be described. The parameter generation unit 303 acquires the determination result of the determination unit 302 and, while the determination result includes the information that smoke is generated in the input image, the parameter generation unit 303 generates a parameter setting the smoke removal process as “on”.

The parameter generation unit 303 specifies the intensity level based on an “intensity-level specifying table (illustration omitted)” which defines the relation between the generation amount of smoke included in the determination result and intensity levels. It is assumed that, the intensity-level specifying table is set in advance so that the higher the generation amount of smoke, the higher the intensity level. The parameter generation unit 303 sets the intensity level as the parameter.

Also, the parameter generation unit 303 sets, in the parameter, the information of the smoke generated region included in the determination result as the information of the region serving as a target of the smoke removal process.

The parameter generation unit 303 sets the smoke removal process as “on” as described above and outputs the parameters, in which the information of the intensity level and the region serving as the target of the smoke removal process is set, to the smoke-removal processing unit 304.

((Smoke-Removal Processing Unit 304))

The smoke-removal processing unit 304 is a processing unit which generates a smoke-removed image, which is the input image from which the smoke has been removed, based on the parameters. In the third embodiment, the smoke-removed image corresponds to the output image. The smoke-removal processing unit 304 outputs the smoke-removed image (output image) to the display device 5155. Also, the smoke-removal processing unit 304 registers the smoke-removed image in the storage unit 301.

If “off” is set for the smoke removal process in the parameters, the smoke-removal processing unit 304 outputs the unchanged input image without executing the smoke removal process.

If “on” is set for the smoke removal process in the parameters, the smoke-removal processing unit 304 executes the following smoke removal process.

The smoke-removal processing unit 304 divides the input image into plural blocks, compares the information of the region serving as the target of the smoke removal process with the blocks, and selects the block serving as the target of the smoke removal process. With respect to the selected block, the smoke-removal processing unit 304 executes a process corresponding to the deterioration estimation unit 31 and a process corresponding to the deterioration correction unit 32 as well as the smoke-removal processing unit 304 of the first embodiment.

Herein, when the pixel values of the input image are converted to the pixel values of the smoke-removed image, the smoke-removal processing unit 304 limits the width of the contrast that allows changes depending on the intensity level. If changes in the pixel values of the smoke-removed image with respect to the pixel values of the input image exceed the width of the permissible contrast, the smoke-removal processing unit 304 carries out adjustment so that the pixel values of the pixels of the smoke-removed image are within the width of the permissible contrast. It is assumed that the relation between the intensity level and the width of the permissible contrast is set in a “contrast specifying table (illustration omitted)” in advance. In the contrast specifying table, the higher the intensity level, the wider the contrast.

«4.3. Effects of Information Processing Device according to Third Embodiment»

According to the information processing device 300 according to the third embodiment of the present disclosure, the parameters for carrying out the smoke removal process are generated based on the used device information and the input information. Since the parameters are optimized by the parameter generation unit 303, the output image obtained by appropriately eliminating smoke from the input image can be generated by executing the smoke removal process by using the parameters.

### 5. FOURTH EMBODIMENT

«5.1. Configuration of System according to Fourth Embodiment»

Next, a fourth embodiment of the present disclosure will be described in detail. A system configuration according to the fourth embodiment of the present disclosure is similar to the system configuration according to the first embodiment of the present disclosure described in FIG. 2. In the following descriptions, an information processing device according to the fourth embodiment will be described as an information processing device 400. Although illustration is omitted, the image capturing device 10, the display device 5155, and the information processing device 400 are mutually connected via the network 20.

Note that the information processing device 400 is assumed to treat smoke or mist as smoke without discriminating between smoke and mist.

«5.2. Configuration of Information Processing Device according to Fourth Embodiment»

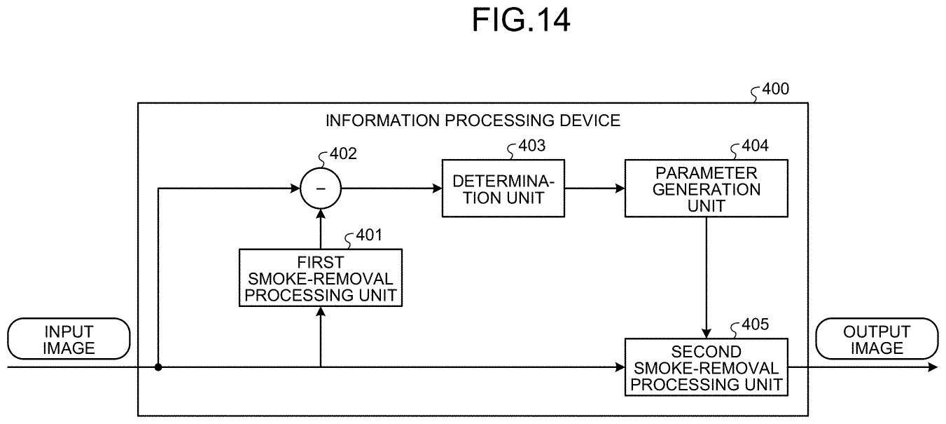

Next, the information processing device 400 according to the fourth embodiment of the present disclosure will be described in detail. FIG. 14 is a diagram illustrating a configuration example of the information processing device according to the fourth embodiment of the present disclosure. As illustrated in FIG. 14, the information processing device 400 has a first smoke-removal processing unit 401, a subtraction unit 402, a determination unit 403, a parameter generation unit 404, and a second smoke-removal processing unit 405.

Every time the input image is received from the image capturing device 10, the information processing device 400 inputs the input image to each of the first smoke-removal processing unit 401 and the subtraction unit 402. Although illustration is omitted in FIG. 14, it is assumed that the information processing device 400 has a communication unit which carries out information communication with the image capturing device 10 and the display device 5155 via the network 20.

((First Smoke-Removal Processing unit 401))

The first smoke-removal processing unit 401 is a processing unit which generates a smoke-removed image, which is the input image from which the smoke has been removed, based on initial parameters set in advance. The first smoke-removal processing unit 401 outputs the smoke-removed image to the subtraction unit 402. The process in which the first smoke-removal processing unit 401 generates the smoke-removed image by using the parameters (initial parameters) is similar to the process of the smoke-removal processing unit 304 according to the third embodiment.

((Subtraction Unit 402))

The subtraction unit 402 is a processing unit which generates a difference image of the input image and the smoke-removed image. For example, the subtraction unit 402 generates the difference image by subtracting the smoke-removed image from the input image. The subtraction unit 402 outputs the information of the difference image to the determination unit 403.

((Determination Unit 403))

The determination unit 403 is a processing unit which determines whether smoke is included in the input image or not based on the difference image. If it is determined that the smoke is included, the determination unit 403 determines the generation amount of the smoke.

An example of a process in which the determination unit 403 determines whether the input image includes smoke or not will be described. For example, the determination unit 403 totals the pixel values of the pixels of the difference image and, if the totaled pixel value is equal to or higher than a threshold value Th1, determines that the input image includes the smoke. It is assumed that the threshold value Th1 is set in advance.

When the determination unit 403 determines that the input image includes smoke, the determination unit 403 executes a process of determining the generation amount of the smoke and a process of specifying a smoke generated region.

For example, the determination unit 403 divides the difference image into plural blocks and calculates the total value of the pixel values for each block. The determination unit 403 specifies, as the smoke generated region, the block in which the total value of the pixel values becomes a threshold value Th2 or higher among the plural blocks.

The determination unit 403 specifies the generation amounts of the smoke based on a “generation-amount specifying table (illustration omitted)”, which defines the relation between the rate of blocks of smoke generated regions to all the blocks of the input image, the total values of the pixel values of the blocks, and the generation amounts of smoke. It is assumed that the information of the generation-amount specifying table is set in advance.

The determination unit 403 outputs the determination result to the parameter generation unit 404. The determination result includes the information on whether smoke has been generated in the input image or not. If the smoke has been generated, the determination result further includes the information on the generation amount of the smoke and the generated region of the smoke.

((Parameter Generation Unit 404))

The parameter generation unit 404 is a processing unit which generates parameters of a smoke removal process based on the determination result of the determination unit 403. The parameters generated by the parameter generation unit 404 includes the information of on/off timing of the smoke removal process, the intensity level of the smoke removal process, and a region serving as a target of the smoke removal process. The process in which the parameter generation unit 404 generates the parameters is similar to the parameter generation unit 303 described in the fourth embodiment. The parameter generation unit 404 outputs the parameters to the second smoke-removal processing unit 405.

((Second Smoke-Removal Processing Unit 405))

The second smoke-removal processing unit 405 is a processing unit which generates a smoke-removed image, which is the input image from which the smoke has been removed, based on the parameters. The second smoke-removal processing unit 405 outputs the smoke-removed image (output image) to the display device 5155. The process in which the second smoke-removal processing unit 405 generates the smoke-removed image by using the parameters is similar to the process of the smoke-removal processing unit 304 according to the third embodiment.

«5.3. Effects of Information Processing Device according to Fourth Embodiment»

According to the information processing device 400 according to the fourth embodiment of the present disclosure, the smoke-removed image is once generated by the initial parameters, the difference image of the input image and the smoke-removed image is generated, and the generation amount and the generated region of the smoke are determined based on the difference image. The information processing device 400 can optimize the parameters of the smoke removal process by using the determination result and generate the output image, which is obtained by appropriately eliminating the smoke from the input image, by executing the smoke removal process by using the parameters.

### 6. FIFTH EMBODIMENT

«6.1. Configuration of System according to Fifth Embodiment»

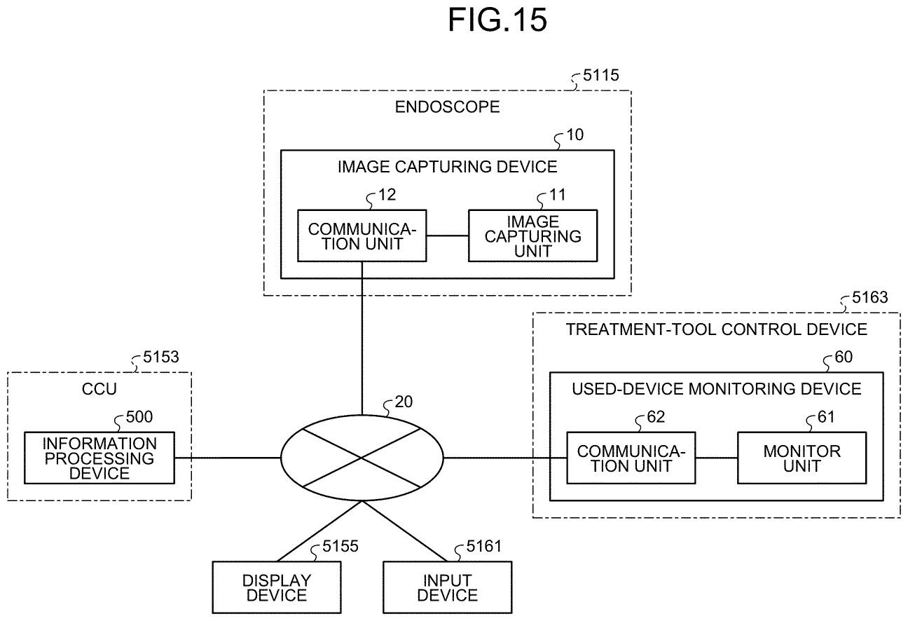

Next, a fifth embodiment of the present disclosure will be described in detail. FIG. 15 is a diagram illustrating a system configuration example according to the fifth embodiment of the present disclosure. As illustrated in FIG. 15, this system has the image capturing device 10, the used-device monitoring device 60, the display device 5155, the input device 5161, and an information processing device 500. The image capturing device 10, the used-device monitoring device 60, the display device 5155, the input device 5161, and the information processing device 500 are mutually connected via the network 20.

The descriptions about the image capturing device 10, the used-device monitoring device 60, and the display device 5155 are similar to the descriptions about the image capturing device 10, the used-device monitoring device 60, and the display device 5155 described in FIG. 11.

The input device 5161 is an input interface for the endoscope operation system 5113. For example, the user operates the input device 5161 to designate a region from which the smoke is to be removed. The information of the region designated by the user from which the smoke is to be removed will be described as “designation information”. The input device 5161 transmits the designation information to the information processing device 500 via the network 20.

Also, the input device 5161 may have a camera and detect a visual line position of the user. The sensing information including the information of the visual line position of the user is transmitted to the information processing device 500 via the network 20.

«6.2. Configuration of Information Processing Device according to Fifth Embodiment»

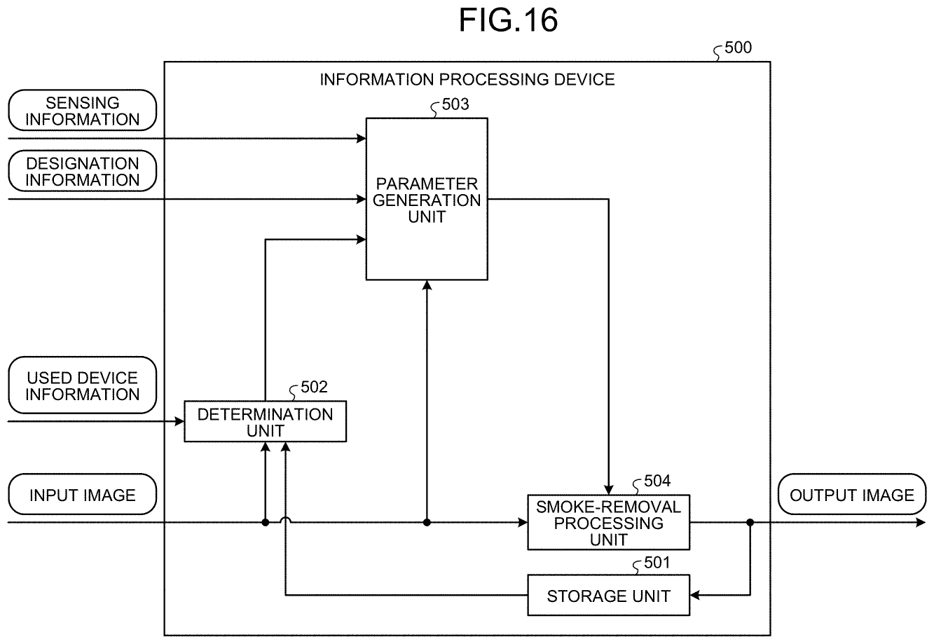

Next, the information processing device 500 according to the fifth embodiment of the present disclosure will be described in detail. FIG. 16 is a diagram illustrating a configuration example of the information processing device according to the fifth embodiment of the present disclosure. As illustrated in FIG. 16, the information processing device 500 has a storage unit 501, a determination unit 502, a parameter generation unit 503, and a smoke-removal processing unit 504.

Every time the input image is received from the image capturing device 10, the information processing device 500 inputs the input image to each of the determination unit 502, the parameter generation unit 503, and the smoke-removal processing unit 504. Every time the used device information is received from the used-device monitoring device 60, the information processing device 300 inputs the used device information to the determination unit 502. Every time the designation information and the sensing information is received from the input device 5161, the information processing device 500 outputs the designation information and the sensing information to the parameter generation unit 503. Although illustration is omitted in FIG. 16, it is assumed that the information processing device 500 has a communication unit which carries out information communication with the image capturing device 10, the used-device monitoring device 60, the input device 5161, and the display device 5155 via the network 20. Note that the information processing device 500 is assumed to treat smoke or mist as smoke without discriminating between smoke and mist.

((Storage Unit 501))

The storage unit 501 is a storage device which stores the information of the latest output image generated by the smoke-removal processing unit 504. The output image is an image in which smoke has not been generated (or smoke has been removed). The output image stored in the storage unit 501 is updated every time a new output image is output from the smoke-removal processing unit 504.

The storage unit 501 corresponds to a semiconductor memory element such as a RAM, a ROM, or a flash memory or a storage device such as a HDD. The storage unit 501 may be either one of a volatile memory and a non-volatile memory or may use both of them.

((Determination Unit 502))

The determination unit 502 is a processing unit which determines whether the input image includes smoke or not based on the used device information and the input image. If it is determined that the smoke is included, the determination unit 502 determines the generation amount of the smoke. The process of the determination unit 502 is similar to the process of the determination unit 302 described in the third embodiment.

The determination unit 502 outputs the determination result to the parameter generation unit 503. The determination result includes the information on whether smoke has been generated in the input image or not. If the smoke has been generated, the determination result further includes the information on the generation amount of the smoke and the generated region of the smoke.

((Parameter Generation Unit 503))

The parameter generation unit 503 is a processing unit which generates parameters of a smoke removal process based on the determination result of the determination unit 502, the designation information, and the sensing information. The parameters generated by the parameter generation unit 503 includes the information of on/off timing of the smoke removal process, the intensity level of the smoke removal process, and a region serving as a target of the smoke removal process.

An example of a process of the parameter generation unit 503 while the determination result includes the information that smoke is not generated in the input image will be described. The parameter generation unit 503 acquires the determination result of the determination unit 502 and, while the determination result includes the information that smoke is not generated in the input image, the parameter generation unit 503 generates a parameter setting the smoke removal process as “off” and outputs the parameter to the smoke-removal processing unit 504.

Note that, while the information that smoke has not been generated in the input image is included, the parameter generation unit 503 does not set, as parameters, the information of the intensity level of the smoke removal process and the region serving as a target of the smoke removal process.

Subsequently, an example of a process of the parameter generation unit 503 while the determination result includes the information that smoke is generated in the input image will be described. The parameter generation unit 503 acquires the determination result of the determination unit 403 and, while the determination result includes the information that smoke is generated in the input image, the parameter generation unit 503 generates a parameter setting the smoke removal process as “on”.

The parameter generation unit 503 specifies the intensity level based on the “intensity-level specifying table (illustration omitted)” which defines the relation between the generation amount of smoke included in the determination result and intensity levels. It is assumed that, the intensity-level specifying table is set in advance so that the higher the generation amount of smoke, the higher the intensity level. The parameter generation unit 503 sets the intensity level as the parameter.

Herein, an example of the process in which the parameter generation unit 503 specifies the region serving as a target of the smoke removal process will be described. The parameter generation unit 503 specifies, as the region to be the target of the smoke removal process, the region which is a smoke generated region included in the determination result and a partial region of the input image set in advance. In the following descriptions, the partial region of the input image set in advance will be described as a “focus region”.

The parameter generation unit 503 may set the focus region in any manner. The parameter generation unit 503 may set the focus region at a center part of the input image. Also, if the designation information is received, the parameter generation unit 503 sets the focus region based on the designation information. If the sensing information is received, the parameter generation unit 503 sets the focus region based on the visual line position of the user.

The parameter generation unit 503 may specify the position of an organ or an operation tool based on the input image and set the focus region based on the specified position of the organ or the operation tool. The parameter generation unit 503 may specify the position of the organ or the operation tool by using any of conventional techniques. For example, the parameter generation unit 503 extracts an edge from the input image, carries out matching by using a template defining predetermined organs or the shapes of operation tools, and specifies the position of the organ or the operation tool.

By executing the above described process, the parameter generation unit 503 sets, as the parameters, the information of the on/off timing of the smoke removal process, the intensity level of the smoke removal process, and the region serving as the target of the smoke removal process and outputs the information to the smoke-removal processing unit 504.

((Smoke-Removal Processing Unit 504))

The smoke-removal processing unit 504 is a processing unit which generates a smoke-removed image, which is the input image from which the smoke has been removed, based on the parameters. The smoke-removal processing unit 504 outputs the smoke-removed image (output image) to the display device 5155. The process in which the smoke-removal processing unit 504 generates the smoke-removed image by using the parameters is similar to the process of the smoke-removal processing unit 304 according to the third embodiment.

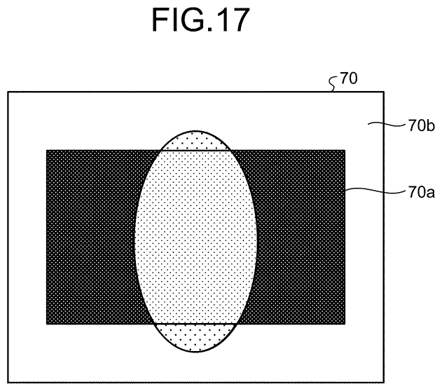

Note that the region serving as the target of the smoke removal process is the smoke generated region included in the determination result and serves as the region which is the focus region. FIG. 17 is a diagram illustrating an example of the output image generated by the smoke removal process according to the fifth embodiment of the present disclosure. As illustrated in FIG. 17, an output image 70 includes a region 70a which has undergone a smoke removal process and a region 70b which has not undergone a smoke removal process.

«6.3. Effects of Information Processing Device according to Fifth Embodiment»

According to the information processing device 500 according to the fifth embodiment of the present disclosure, the region serving as the target of the smoke removal process is limited to the smoke generated region included in the determination result and the region which serves as the focus region. By virtue of this, the smoke removal process is executed only for the focus region. Therefore, the smoke can be removed from the part important in the operation, and the user can easily see whether the process of smoke removal is working or not. Also, erroneous removal of the things other than smoke can be prevented.

### 7. SIXTH EMBODIMENT

«7.1. Configuration of System according to Sixth Embodiment»

Next, a sixth embodiment of the present disclosure will be described in detail. A system configuration according to the sixth embodiment of the present disclosure is similar to the system configuration according to the first embodiment of the present disclosure described in FIG. 2. In the following descriptions, an information processing device according to the sixth embodiment will be described as an information processing device 600. Although illustration is omitted, the image capturing device 10, the display device 5155, and the information processing device 400 are mutually connected via the network 20.

Note that the information processing device 600 is assumed to treat smoke or mist as smoke without discriminating between smoke and mist.

«7.2. Configuration of System according to Sixth Embodiment»

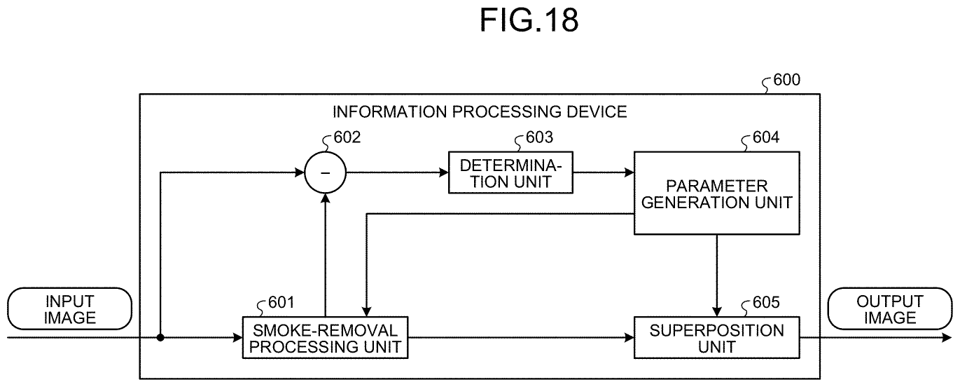

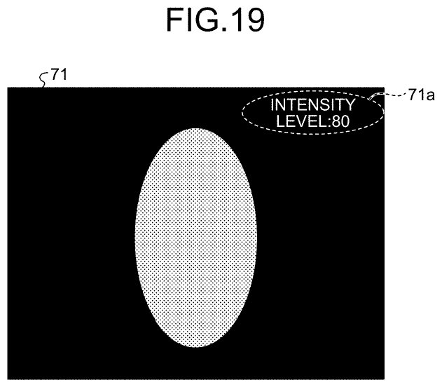

Next, the information processing device 600 according to the sixth embodiment of the present disclosure will be described in detail. FIG. 18 is a diagram illustrating a configuration example of the information processing device according to the sixth embodiment of the present disclosure. As illustrated in FIG. 18, the information processing device 600 has a smoke-removal processing unit 601, a subtraction unit 602, a determination unit 603, a parameter generation unit 604, and a superposition unit 605.

Every time the input image is received from the image capturing device 10, the information processing device 600 inputs the input image to each of the smoke-removal processing unit 601 and the subtraction unit 402. Although illustration is omitted in FIG. 18, it is assumed that the information processing device 600 has a communication unit which carries out information communication with the image capturing device 10 and the display device 5155 via the network 20.

((Smoke-Removal Processing Unit 601))

The smoke-removal processing unit 601 is a processing unit which generates a smoke-removed image, which is the input image from which the smoke has been removed, based on the parameters acquired from the parameter generation unit 604. The smoke-removal processing unit 601 outputs the smoke-removed image to the subtraction unit 602 and the superposition unit 605. The process in which the smoke-removal processing unit 601 generates the smoke-removed image by using the parameters is similar to the process of the smoke-removal processing unit 304 according to the third embodiment.

((Subtraction Unit 602))

The subtraction unit 602 is a processing unit which generates a difference image of the input image and the smoke-removed image. For example, the subtraction unit 602 generates the difference image by subtracting the smoke-removed image from the input image. The subtraction unit 602 outputs the information of the difference image to the determination unit 603.

((Determination Unit 603))

The determination unit 603 is a processing unit which determines whether smoke is included in the input image or not based on the difference image. If it is determined that the smoke is included, the determination unit 603 determines the generation amount of the smoke. The process of the determination unit 603 is similar to the process of the determination unit 403 according to the fourth embodiment.

The determination unit 603 outputs the determination result to the parameter generation unit 604. The determination result includes the information on whether smoke has been generated in the input image or not. If the smoke has been generated, the determination result further includes the information on the generation amount of the smoke and the generated region of the smoke.

((Parameter Generation Unit 604))

The parameter generation unit 604 is a processing unit which generates parameters of a smoke removal process based on the determination result of the determination unit 603. The parameters generated by the parameter generation unit 604 includes the information of on/off timing of the smoke removal process, the intensity level of the smoke removal process, and a region serving as a target of the smoke removal process. The process in which the parameter generation unit 604 generates the parameters is similar to the parameter generation unit 303 described in the fourth embodiment. The parameter generation unit 604 outputs the parameters to the smoke-removal processing unit 601.

Also, the parameter generation unit 604 outputs the information of the intensity level of the smoke removal process to the superposition unit 605.

((Superposition Unit 605))

The superposition unit 605 is a processing unit which superposes the information of the intensity level of the smoke removal process on the output image. FIG. 19 is a diagram for describing a process of the superposition unit according to the sixth embodiment of the present disclosure. In the example illustrated in FIG. 19, information 71a which is “INTENSITY LEVEL: 80” is superposed on the output image 71. The superposition unit 605 outputs the output image of the processing result to the display device 5155.

«7.3. Effects of Information Processing Device according to Sixth Embodiment»

According to the information processing device 600 according to the sixth embodiment of the present disclosure, the information of the intensity level of the smoke removal process is superposed on the output image. By virtue of this, the user can see whether the smoke removal process is working or not. Moreover, by displaying the numerical value of the intensity level, the user can see the degree of the smoke generation amount and the degree of the effect of smoke removal.

### 8. HARDWARE CONFIGURATION

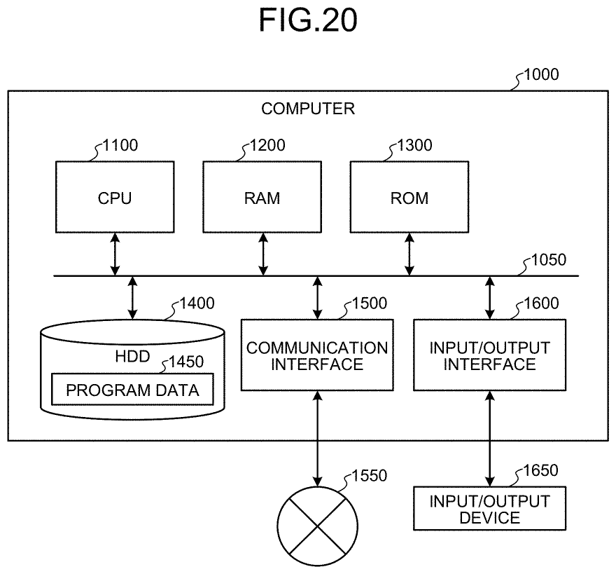

The information processing devices according to the above described embodiments are realized, for example, by a computer 1000 having a configuration as illustrated in FIG. 20. Hereinafter, the information processing device 100 according to the first embodiment will be used as an example for description. FIG. 20 is a hardware configuration diagram illustrating an example of a computer 1000 which realizes the functions of the information processing device. The computer 1000 has a CPU 1100, a RAM 1200, a read only memory (ROM) 1300, a hard disk drive (HDD) 1400, a communication interface 1500, and an input/output interface 1600. Each part of the computer 1000 is connected by a bus 1050.

The CPU 1100 operates based on programs stored in the ROM 1300 or the HDD 1400 and controls each part. For example, the CPU 1100 deploys the programs, which are stored in the ROM 1300 or the HDD 1400, in the RAM 1200 and executes processing corresponding to the various programs.

The ROM 1300 stores, for example, a boot program such as Basic Input Output System (BIOS), which is executed by the CPU 1100 upon startup of the computer 1000, and a program dependent on hardware of the computer 1000.

The HDD 1400 is a computer-readable recording medium which non-temporarily records, for example, programs executed by the CPU 1100 and data used by the programs. Specifically, the HDD 1400 is a recording medium which records the information processing program according to the present disclosure serving as an example of program data 1450.

The communication interface 1500 is an interface for connecting the computer 1000 to an external network 1550 (for example, the Internet). For example, the CPU 1100 receives data from other equipment and transmits the data generated by the CPU 1100 to other equipment via the communication interface 1500.

The input/output interface 1600 is an interface for connecting an input/output device 1650 and the computer 1000. For example, the CPU 1100 receives data from an input device such as a keyboard or a mouse via the input/output interface 1600. Also, the CPU 1100 transmits data to an output device such as a display, a speaker, or a printer via the input/output interface 1600. Also, the input/output interface 1600 may function as a media interface, which reads a program or the like recorded in a predetermined recording medium (media). The media are, for example, optical recording media such as digital versatile discs (DVDs) and phase change rewritable disks (PDs), magnetooptical recording media such as magneto-optical disks (MOs), tape media, magnetic recording media, or semiconductor memories.

For example, if the computer 1000 functions as the information processing device 100 according to the first embodiment, the CPU 1100 of the computer 1000 realizes functions of the determination unit 102, the smoke-removal processing unit 103, the mist-removal processing unit 104, the generation unit 105, etc. by executing the information processing program loaded on the RAM 1200. Moreover, the HDD 1400 stores the generation program according to the present disclosure and the data in the storage unit 101. The CPU 1100 reads the program data 1450 from the HDD 1400 to execute the data, but may acquire these programs from other devices via the external network 1550 as another example.

### 9. CONCLUSION

An information processing device has a generation unit. The generation unit that acquires an input image serving as an intraoperative image and generates an output image based on whether the input image includes an intraoperatively generated matter or not. Also, the information processing device has a determination unit. The determination unit determines whether the input image includes smoke or mist or not. The determination unit further determines the generation amount of the smoke or the mist based on the input image. The determination unit further determines the ratio of the smoke and the mist based on the input image. The generation unit is characterized by eliminating influence of the smoke or the mist from an entirety of the output image. By virtue of this, even if smoke or mist is generated during an operation of an endoscope operation and deteriorates view, clearer view can be ensured by using the information processing device regardless of the amounts of the generated smoke or mist.

The determination unit determines whether the input image includes the smoke or the mist or not by further using a type and an operation status of an electronic device connected to the information processing device. By virtue of this, the determination precision on whether the input image includes smoke or mist or not can be improved.

The information processing device further has a smoke-removal processing unit which generates a smoke-removed image which is the input image from which the smoke has been removed. The generation unit generates the output image by using the determination result, the input image, and the smoke-removed image. By virtue of this, the smoke-removed image from which the smoke has been removed can be generated, and the output image without generation of smoke can be generated by using this smoke-removed image.

The smoke-removal processing unit estimates deterioration of the input image based on the output image and the input image and generates the smoke-removed image based on a result of the estimation. By virtue of this, the smoke included in the input image can be appropriately eliminated.

The information processing device further has a superposition unit which superposes, on the output image, the information about the smoke removed by the smoke-removal processing unit. Herein, the information about the smoke may be any information as long as the information relates to the reduced smoke like presence/absence of execution of a smoke reduction process, the reduced degree of the smoke, and the intensity of the smoke reducing process. By virtue of this, the user can see whether the smoke removal process is working or not. Moreover, by displaying the numerical value of the intensity level, the user can see the degree of the smoke generation amount and the degree of the effect of smoke removal.

The information processing device further has a subtraction unit that generates a difference image between the input image and the smoke-removed image; wherein the determination unit specifies a generation amount of the smoke based on the difference image and, based on the generation amount, generates information about the smoke removed by the smoke-removal processing unit. Also, the information processing device further has a parameter generation unit that generates a parameter used in a smoke removal process based on the determination result of the determination unit, wherein the smoke-removal processing unit generates, based on the parameter, a smoke-removed image obtained by removing the smoke from the input image. The parameter generation unit generates, based on the determination result of the determination unit, the parameter including timing of start or end of a smoke removal process, an intensity of the smoke removal process, and/or a target region of the smoke removal process. In this manner, the parameters of the smoke removal process can be optimized by using the difference image, and the output image, which is obtained by appropriately eliminating the smoke from the input image, can be generated by executing the smoke removal process by using the parameters.

The information processing device further has a mist-removal processing unit that generates a mist-removed image obtained by removing the mist from the input image; wherein the generation unit generates the output image by using the determination result, the input image, and the mist-removed image. The mist-removal processing unit specifies a generated region of the mist based on the input image, generates a corrected image including the generated region of the mist corrected according to information of a region around the generated region of the mist, estimates deterioration of the corrected image based on the corrected image and the output image, and generates the mist-removed image based on a result of the estimation. By adjusting the contrast of the input image in two levels in this manner, mist having an uneven pattern which has different characteristics from smoke can be appropriately eliminated from the input image.

The generation unit generates the output image by synthesizing the input image, the smoke-removed image, and the mist-removed image based on the generation amount and a ratio of the smoke and the mist. By virtue of this, the smoke-removed image and the mist-removed image can be synthesized by the blend ratio α, which is based on the generation probabilities of smoke and mist, and the smoke and the mist included in the input image can be appropriately eliminated.

The generation unit is characterized by eliminating influence of the smoke or the mist from a partial region of the output image. By virtue of this, the smoke removal process is executed only for the focus region. Therefore, the smoke can be removed from the part important in the operation, and the user can easily see whether the process of smoke removal is working or not. Also, erroneous removal of the things other than smoke can be prevented.

The generation unit specifies the partial region based on a position of an organ or an operation tool specified from the input image and eliminates the influence of the smoke or the mist from the partial region of the output image. The generation unit specifies the partial region based on a point-of-view position of a user and eliminates the influence of the smoke or the mist from the partial region of the output image. By virtue of this, the region on which the user focuses can be cleared.

The effects described in the present description are merely examples and are not limitative, and other effects may be included.

The present technique can also employ following configurations.

(1)

An information processing device including

a generation unit that acquires an input image serving as an intraoperative image and generates an output image based on whether the input image includes an intraoperatively generated matter or not.

(2)

The information processing device according to (1) further including a determination unit that determines whether the input image includes smoke or mist or not.

(3)

The information processing device according to (2), wherein the determination unit determines whether the input image includes the smoke or the mist or not by further using a type and an operation status of an electronic device connected to the information processing device.

(4)

The information processing device according to (2) or (3), wherein the determination unit further determines a generation amount of the smoke or the mist based on the input image.

(5)

The information processing device according to (2), (3) or (4), wherein the determination unit further determines a ratio of the smoke to the mist based on the input image.

(6)

The information processing device according to (4), further including a smoke-removal processing unit that generates a smoke-removed image obtained by removing the smoke from the input image; wherein the generation unit generates the output image by using the determination result, the input image, and the smoke-removed image.

(7)

The information processing device according to (6), wherein the smoke-removal processing unit estimates deterioration of the input image based on the output image and the input image and generates the smoke-removed image based on a result of the estimation.

(8)

The information processing device according to (7), further including a superposition unit that superposes, on the output image, information about the smoke removed by the smoke-removal processing unit.

(9)

The information processing device according to (8), further including a subtraction unit that generates a difference image between the input image and the smoke-removed image; wherein the determination unit specifies a generation amount of the smoke based on the difference image and, based on the generation amount, generates information about the smoke removed by the smoke-removal processing unit.

(10)

The information processing device according to (6), (7) or (8), further including a mist-removal processing unit that generates a mist-removed image obtained by removing the mist from the input image; wherein the generation unit generates the output image by using the determination result, the input image, and the mist-removed image.

(11)

The information processing device according to (10), wherein the mist-removal processing unit specifies a generated region of the mist based on the input image, generates a corrected image including the generated region of the mist corrected according to information of a region around the generated region of the mist, estimates deterioration of the corrected image based on the corrected image and the output image, and generates the mist-removed image based on a result of the estimation.

(12)

The information processing device according to (11), wherein the generation unit generates the output image by synthesizing the input image, the smoke-removed image, and the mist-removed image based on the generation amount and a ratio of the smoke and the mist.

(13)

The information processing device according to any one of (6) to (12), further including a parameter generation unit that generates a parameter used in a smoke removal process based on the determination result of the determination unit, wherein the smoke-removal processing unit generates, based on the parameter, a smoke-removed image obtained by removing the smoke from the input image.

(14)

The information processing device according to (13), wherein the parameter generation unit generates, based on the determination result of the determination unit, the parameter including timing of start or end of a smoke removal process, an intensity of the smoke removal process, and/or a target region of the smoke removal process.

(15)

The information processing device according to any one of (2) to (14), wherein the generation unit eliminates influence of the smoke or the mist from an entirety of the output image.

(16)

The information processing device according to any one of (2) to (14), wherein the generation unit eliminates influence of the smoke or the mist from a partial region of the output image.

(17)

The information processing device according to (16), wherein the generation unit specifies the partial region based on a position of an organ or an operation tool specified from the input image and eliminates the influence of the smoke or the mist from the partial region of the output image.

(18)

The information processing device according to (16), wherein the generation unit specifies the partial region based on a point-of-view position of a user and eliminates the influence of the smoke or the mist from the partial region of the output image.

(19)

A generation method of causing a computer to execute a process of

acquiring an input image serving as an intraoperative image and

generating an output image based on whether the input image includes an intraoperatively generated matter or not.

(20)

A generation program for causing a computer to function as

a generation unit that acquires an input image serving as an intraoperative image and generates an output image based on whether the input image includes an intraoperatively generated matter or not.

### REFERENCE SIGNS LIST

10 IMAGE CAPTURING DEVICE

11 IMAGE CAPTURING UNIT

12, 62 COMMUNICATION UNIT

20 NETWORK

31, 43 DETERIORATION ESTIMATION UNIT

32 DETERIORATION CORRECTION UNIT

41 GENERATION-REGION SPECIFYING UNIT

42 FIRST DETERIORATION CORRECTION UNIT

44 SECOND DETERIORATION CORRECTION UNIT

51 FIRST BLEND-RATIO CALCULATING UNIT

52 FIRST BLEND PROCESSING UNIT

53 SECOND BLEND-RATIO CALCULATING UNIT

54 SECOND BLEND PROCESSING UNIT

60 USED-DEVICE MONITORING DEVICE

61 MONITOR UNIT

100, 200, 300, 400, 500, 600 INFORMATION PROCESSING DEVICE

101, 201, 301, 501 STORAGE UNIT

102, 202, 302, 403, 502, 603 DETERMINATION UNIT

103, 203, 304, 504, 601 SMOKE-REMOVAL PROCESSING UNIT

104, 204 MIST-REMOVAL PROCESSING UNIT

105, 205 GENERATION UNIT

303, 404, 503, 604 PARAMETER GENERATION UNIT

401 FIRST SMOKE—REMOVAL PROCESSING UNIT

402 SUBTRACTION UNIT

405 ECOND SMOKE—REMOVAL PROCESSING UNIT

605 SUPERPOSITION UNIT

1. An information processing device including
a generation unit that acquires an input image serving as an intraoperative image and generates an output image based on whether the input image includes an intraoperatively generated matter or not.

2. The information processing device according to claim 1 further including a determination unit that determines whether the input image includes smoke or mist or not.

3. The information processing device according to claim 2, wherein the determination unit determines whether the input image includes the smoke or the mist or not by further using a type and an operation status of an electronic device connected to the information processing device.

4. The information processing device according to claim 2, wherein the determination unit further determines a generation amount of the smoke or the mist based on the input image.

5. The information processing device according to claim 2, wherein the determination unit further determines a ratio of the smoke to the mist based on the input image.

6. The information processing device according to claim 4, further including a smoke-removal processing unit that generates a smoke-removed image obtained by removing the smoke from the input image; wherein the generation unit generates the output image by using the determination result, the input image, and the smoke-removed image.

7. The information processing device according to claim 6, wherein the smoke-removal processing unit estimates deterioration of the input image based on the output image and the input image and generates the smoke-removed image based on a result of the estimation.

8. The information processing device according to claim 7, further including a superposition unit that superposes, on the output image, information about the smoke removed by the smoke-removal processing unit.

9. The information processing device according to claim 8, further including a subtraction unit that generates a difference image between the input image and the smoke-removed image;
wherein the determination unit specifies a generation amount of the smoke based on the difference image and, based on the generation amount, generates information about the smoke removed by the smoke-removal processing unit.

10. The information processing device according to claim 6, further including a mist-removal processing unit that generates a mist-removed image obtained by removing the mist from the input image; wherein the generation unit generates the output image by using the determination result, the input image, and the mist-removed image.

11. The information processing device according to claim 10, wherein the mist-removal processing unit specifies a generated region of the mist based on the input image, generates a corrected image including the generated region of the mist corrected according to information of a region around the generated region of the mist, estimates deterioration of the corrected image based on the corrected image and the output image, and generates the mist-removed image based on a result of the estimation.

12. The information processing device according to claim 11, wherein the generation unit generates the output image by synthesizing the input image, the smoke-removed image, and the mist-removed image based on the generation amount and a ratio of the smoke and the mist.

13. The information processing device according to claim 6, further including a parameter generation unit that generates a parameter used in a smoke removal process based on the determination result of the determination unit, wherein the smoke-removal processing unit generates, based on the parameter, a smoke-removed image obtained by removing the smoke from the input image.

14. The information processing device according to claim 13, wherein the parameter generation unit generates, based on the determination result of the determination unit, the parameter including timing of start or end of a smoke removal process, an intensity of the smoke removal process, and/or a target region of the smoke removal process.

15. The information processing device according to claim 2, wherein the generation unit eliminates influence of the smoke or the mist from an entirety of the output image.

16. The information processing device according to claim 2, wherein the generation unit eliminates influence of the smoke or the mist from a partial region of the output image.

17. The information processing device according to claim 16, wherein the generation unit specifies the partial region based on a position of an organ or an operation tool specified from the input image and eliminates the influence of the smoke or the mist from the partial region of the output image.

18. The information processing device according to claim 16, wherein the generation unit specifies the partial region based on a point-of-view position of a user and eliminates the influence of the smoke or the mist from the partial region of the output image.

19. A generation method of causing a computer to execute a process of
acquiring an input image serving as an intraoperative image and
generating an output image based on whether the input image includes an intraoperatively generated matter or not.

20. A generation program for causing a computer to function as
a generation unit that acquires an input image serving as an intraoperative image and generates an output image based on whether the input image includes an intraoperatively generated matter or not.
